(19) United States
(12) Patent Application Publication
(10) Pub. No.: US 2005/0004105 A1
(43) Pub. Date: Jan. 6, 2005

(54) Treatment for a attention-deficit hyperactivity disorder

(76) Inventors: Emer Leahy, Bedford, NY (US); Berend Olivier, Lelytstad (NL)

Correspondence Address: MORGAN & FINNEGAN, L.L.P., 3 WORLD FINANCIAL CENTER, NEW YORK, NY, 10281-2101, US

(21) Appl. No.: 10/767,788

(22) Filed: Jan. 29, 2004

Related U.S. Application Data
(60) Provisional application No. 60/443,457, filed on Jan. 29, 2003.

(51) Int. Cl. A61K 31/55; A61K 31/496; A61K 31/495

(52) U.S. Cl. 514/218; 514/252.13; 514/255.03

(57) ABSTRACT

A method for treating Attention Deficit/Hyperactivity Disorder (ADHD) in humans using a 5-HT_{1A} receptor agonist is provided.

### FIELD OF THE INVENTION

The present invention is directed to a novel method of treating Attention-Deficit/Hyperactivity Disorder (“ADHD”).

### BACKGROUND OF THE INVENTION

Attention-Deficit/Hyperactivity Disorder (ADHD) is a behavior disorder characterized by problems with control of attention and hyperactivity-impulsivity. The attentional difficulties and impulsivity associated with ADHD have been persuasively documented in laboratory investigations using cognitive tasks. Although these problems typically present together, one may be present without the other to qualify for a diagnosis (Am. Psychiatric Assoc. Diagnostic and Statistical Manual of Mental Disorders, 4^{th} Ed., Text Revision, 2000) (DSM-IV-TR). Generally, attention deficit or inattention becomes apparent when a child enters elementary school. A modified form of the disorder can persist into adulthood (Am. Psychiatric Assoc. Diagnostic and Statistical Manual of Mental Disorders, 3^{rd} Ed., 1987). With respect to the attention component, the child is easily distracted by outside stimuli, neglects finishing tasks, and has difficulty maintaining attention. Regarding the activity component, the child is often fidgety, impulsive, and overactive. The symptoms of ADHD may be apparent as young as preschoolers and are virtually always present prior to the age of 7 (Halperin et al., J. Am. Acad. Child Adolescent Psychiatry, 32:1038-1043, 1993).

According to the DSM-IV-TR, diagnostic criteria for Attention-Deficit/Hyperactivity Disorder relate to symptoms associated with inattention and/or hyperactivity-impulsivity. Three subtypes of ADHD are diagnosed based on the predominant symptoms presented.

Many of the symptoms that are characteristic of ADHD occur occasionally in normal children. Children with ADHD, however, exhibit these symptoms frequently, which tends to interfere with the child's day to day functioning. Such children are often challenged by academic underachievement because of excitability and impaired interpersonal relationships.

ADHD affects 2-6% of grade school children. Pediatricians report that approximately 4% of their patients have ADHD; however, in practice the diagnosis is made in children who meet several, but not all of the diagnostic criteria that is recommended in DMS-IV-TR (Wolraich et al., Pediatrics, 86(1):95-101, 1990). Boys are four times more likely to have the disorder than girls and the disorder is found in all cultures (Ross & Ross, Hyperactivity, New York, 1982).

Psychomotor stimulants are the most common treatment for ADHD. Safer & Krager (1988) reported that 99% of the children with ADHD were treated with stimulants, of which 93% were given methylphenidate hydrochloride (Ritalin), and the remainder were given dextroamphetamine sulfate (d-amphetamine) or pemoline (Safer & Krager, J.A.M.A., 260:2256-2258, 1988). Four separate psychostimulant medications consistently reduce the central features of ADHD, particularly the symptoms of inattention and ADHD associated hyperactivity-impulsivity: methylphenidate, d-amphetamine, pemoline, and a mixture of amphetamine salts (Spender et al., Arch. Gen. Psychiatry, 52:434-443, 1995). These drugs block uptake sites for catecholamines on presynaptic neurons or stimulate the release of granular stores of catecholamines. They are metabolized and leave the body fairly rapidly, and have a therapeutic duration of action of 1 to 4 hours. The psychostimulants do not appear, however, to effect long-term changes in social or academic skills (Pelham et al., J. Clin. Child Psychology, 27:190-205, 1998). Stimulants are generally started at a low dose and adjusted weekly. Common stimulant side effects include insomnia, decreased appetite, stomachaches, headaches, and jitteriness. Psychostimulants also have the potential for abuse, because they are addictive. Thus, current methods of treating ADHD provide inadequate treatment for some patients and/or have side effects that limit their usefulness.

Children who cannot tolerate psychostimulants often use the atypical antidepressant bupropion (Buck, Pediatr. Pharmacother. Vol. 8, No. 4, April, 2002). While bupropion is not as effective as stimulants, it may be used as an adjunct to augment stimulant treatment.

Effective pharmacotherapy for ADHD is complicated by the presence of comorbid behavioral disorders, including aggression, impulse control disorders, and depression, which may be relieved by compounds that do not address the core behavioral symptoms of inattentiveness and impulsivity/hyperactivity.

Castellanos et al. concluded that ADHD is a genetically programmed disorder of brain development resulting from altered function of the frontal-striatal-pallidal-thalamocortical loops which regulate cognitive processes, attention, and motor output behaviors (Castellanos et al., Arch. Gen. Psychiatry, 53: 607-616, 1996). Although the precise etiology of ADHD is unknown, neurotransmitter deficits, genetics, and perinatal complications have been implicated.

Individuals with ADHD have been reported to have impairments in their ability to perceive intervals of time (Conners & Levin, Psychopharmacol. Bulletin, 32(1):67-73, 1996). Time perception is a useful measure of cognitive function, sensitive to dopaminergic and cholinergic manipulations in animals and humans. As in all behavioral tasks, several processes underlie good steady state performance in a temporal task. These behavioral tasks include: attention, motivation, short and long term memory, motor coordination, and instrumental learning. Scaling, discrimination, and reproduction are the three main types of temporal tasks that have been identified. In scaling, subjects must, for example, categorize a stimulus into a given set of categories (“that was a long duration”) or verbally estimate the duration (“that was a 4 s duration”). In discrimination, a comparison is made between two durations (“the second stimulus was longer than the first”). Finally, in reproduction, a response is made that bears some relation with the stimulus (e.g. only responses that are as long or longer than the stimulus are correct).

Time perception is a particularly effective measure for testing cognitive deficits in ADHD individuals. For example, Conners & Levin (1996) showed that ADHD adults improve in measures of attention and timing with the administration of nicotine. Nicotine, like the psychostimulants methylphenidate and d-amphetamine, acts as an indirect dopamine agonist and improves attention and arousal. Studies indicate that adults and adolescents with ADHD smoke much more frequently than normal individuals or those with other psychiatric conditions, perhaps as a form of self-medication for ADHD symptoms. The results indicate that there was a significant clinician-rated global improvement, self-rated vigor and concentration, and improved performance on chronometric measures of attention and timing accuracy, and side effects were minimal (Conners & Levin, supra).

At present, seven main 5-HT receptor classes have been identified: 5-HT₁, 5-HT₂, 5-HT₃, 5-HT₄, 5-HT₅, 5-HT₆ and 5-HT₇. Radioligand binding studies have revealed at least five subtypes of the 5-HT₁ receptor (1A, 1B, 1D, 1E and 1F). 5-HT_{1A} receptors are located primarily in hippocampus, entorhinal cortex, septal nuclei and raphé nuclei. 5-HT_{1A} receptors are present presynaptically on 5-HT neurons in the raphé nuclei, where they function as autoreceptors, decreasing the firing rate of 5-HT neurons and decreasing 5-HT turnover (Sprouse and Aghajanian, Eur. J. Pharmacol. 128:295-98, 1986; Sprouse and Aghajanian, Synapse, 1:3-9, 1987; Hamon et al., J. Pharmacol. Exp. Ther., 246:745-52, 1988). In 5-HT terminal fields, 5-HT_{1A} receptors are reported to mediate firing rate of target neurons and the release of neurotransmitters. For example, 5-HT_{1A} receptors have been reported to mediate a decrease in the firing rate of CA1 pyramidal neurons in CA1 of dorsal hippocampus has been reported (see Tada et al., J. Pharmacol. Exp. Ther. 288:843-848, 1999), as well as enhancement of norepinephrine (NE) release in the hippocampus. 5-HT_{1A} receptors are believed to mediate inhibitory signaling through pertussin toxin-sensitive G proteins, which results in inhibition of cAMP accumulation, activation of potassium channels, or inactivation of calcium channels (Peroutka. J. Neurochem., 60:408-416, 1993; Hoyer et al., Pharmacol. Rev., 46:157-203, 1994).

Compounds having 5-HT_{1A} activity in the central nervous system may be categorized, according to well recognized pharmacological principles, as full agonists, partial agonists, and antagonists (see Fletcher et al., Trends Pharmacol. Sci. 14(12):41-8, 1993). 5-HT_{1A} agonists are numerous and include a range of chemical structures, but many possess a piperazine or aryl piperazine core. 5-HT_{1A} full agonists and partial agonists are reported to be useful as antianxiety agents or antidepressants.

The prototypical 5-HT_{1A} full agonist is 8-hydroxy-2(di-n-propylamino)tetralin (8-OH-DPAT), which is reported to have an affinity (Kᵢ, inhibition/displacement constant) of 2.5 nM for 5-HT_{1A} receptors, well above its affinity for α₁ adrenergic receptors (Kᵢ=380 nM) or 5-HT_{1D} receptors (Kᵢ=930 nM) (Schipper, J. et al., 1991, Hum. Psychopharmacol. 6:S53-61, 1991). The affinity of 8-OH-DPAT for other neurotransmitter receptors (Kᵢ>1000 nM; Schipper et al., 1991, supra) is functionally inconsequential. In addition to having a selective affinity for 5-HT_{1A} receptors, 8-OH-DPAT produces biochemical, electrophysiological and behavioral effects that are employed as a standard by which 5-HT_{1A} ligands are functionally characterized as agonists. For example, 8-OH-DPAT inhibits 5-HT dorsal raphé neuron firing (Sprouse and Aghajanian, 1986; 1987, supra), induces hypothermia (Hjorth, J. Neural Transm. 61: 131-35, 1985) and spontaneous tail flicks (Millan et al. J. Pharmacol. Exp. Ther. 256:973-82, 1991), inhibits forskolin-induced cAMP production (Pauwels et al., Biochem. Pharmacol., 45(2):375-83, 1993) and stimulates corticosterone secretion (Przegalinski et al., Pharmacol. Biochem. Behav., 33:329-43, 1989). The clinical usefulness of 8-OH-DPAT is limited, however, by its extremely short half-life and poor oral availability.

Flesinoxan, a phenylpiperazine derivative [(+)(4-fluoro-N-[2-[4-[2-(hydroxymethyl)-1,4-benzodioxane-5-yl] 1-piperazinyl]ethyl]benzamide) HCl], is a potent and selective 5-HT_{1A} full agonist (Van Wijngaarden et al., Eur. J. Pharmacol. 188:301-312, 1990). The selectivity of flesinoxan for 5-HT_{1A} receptors is well-documented. Flesinoxan is reported to have Kᵢ of 1.7 nM for 5-HT_{1A} receptors, compared to the functionally lower affinity for 5-HT_{1D} (Kᵢ=160 nM) and dopamine D₂ (Kᵢ=140 nM) receptors, and an even lower affinity for α₁ adrenergic receptors (Kᵢ=380 nM), where it acts as an antagonist, and 5-HT_{1B} receptors (Kᵢ=810 nM) (Schipper, J. et al., 1991, supra; Boddeke et al. Naunyn-Schmied. Arch. Pharmacol., 345:257-263, 1992). In a two-lever operant drug discrimination procedure, in which rats were trained to discriminate flesinoxan (0.5 mg/kg i.p.) from saline, flesinoxan did not generalize to the stimuli of an α₁ adrenoceptor antagonist, α₂ adrenoceptor agonist, dopamine receptor agonist or antagonists (Ybema et al., Eur. J. Pharmacol., 256(2): 141-7, 1994). Flesinoxan exhibits the functional characteristics of a 5-HT_{1A} agonist, including inhibition of forskolin-stimulated cAMP production (Schoeffler and Hoyer, Brit. J. Pharmacol., 95:975-85, 1988), induction of hypothermia (Hadrava et al., Neuropharmacol., 34(10):1311-26, 1995; Seletti et al., Neuropharmacol., 13(2):93-104, 1995), and inhibition of 5-HT neuronal firing rate in the dorsal raphé (Hadrava et al., 1995, supra; Lejeune and Millan, Synapse, 30:172-80, 1998).

Flesinoxan was developed as an antihypertensive agent (EP0138280). Flesinoxan, like other 5-HT_{1A} agonists, has been described as useful in the treatment of anxiety and depression (EP0307061; Grof et al., Int. Clin. Psychopharmacol 83:167-72, 1993; Bradford and Stevens, Am Coll Neuropsychopharmacol. (Abstr. 167), 1994). Flesinoxan also has been shown to enhance word and picture recall, word recognition, and reaction times. These improvements were apparent only after a few days of dosing, however, and were most pronounced in elderly subjects, 75 years of age and older (EP710481A1). Therefore, these “cognitive enhancement” effects are likely related to improved memory rather than to effects on inattention or impulsivity. Moreover, the tests used are not relevant to DSM-IV-TR ADHD diagnostics. Note that impulsivity in ADHD is characterized by impatience and difficulty delaying response (DSM-IV-TR at 86). Another 5-HT_{1A} agonist, lesopitron, has been suggested to be useful as a “cognitive enhancer” for treatment of dementia, memory dysfunction, and Alzheimer's disease (U.S. Pat. No. 5,182,281).

The azapirone derivative buspirone is a partial 5-HT_{1A} agonist that also has significant affinity for dopamine D₂ receptors, where it acts as an antagonist. Thus, unlike flesinoxan, buspirone will functionally bind to both 5-HT_{1A} and D₂ receptors at the same concentration. In addition, the major metabolite of buspirone is active as an antagonist at adrenergic α₂ receptors. 5-HT_{1A} partial agonists have been suggest to have therapeutic potential in the treatment of impulse control disorders, depression and alcohol abuse (van Hest, Psychopharmacol., 107: 474, 1992; Schipper et al., 1991, supra, 1991; Cevro et al., Eur. J. Pharmacol., 158:53, 1988; Glitz and Pohl, Drugs, 41:11, 1991). Because these drugs have effects at numerous receptors, however, especially including NE and DA receptors, the mechanism of such effects is unclear, and likely complex.

Buspirone has been suggested as effective for the treatment of ADHD, however its beneficial effects are thought to be mediated through its unique ability to increase NE and DA output (Malhotra et al., J. Am. Acad. Child Adolesc. Psychiatr. 37(4):364-371, 1998). Buspirone's actions on 5-HT system were suggested to be useful in controlling behavioral disruptions, such as aggression and mood disruptions, Which symptoms are related to conduct disorders sometimes comorbid with ADHD (Malhotra et al., 1998, supra). Buspirone also has been asserted to be useful in the treatment of ADHD because it shares some of the electrophysiological properties of stimulants d-amphetamine and methylphenidate, without concomitant motor stimulation (EP0497314; Balon, J. Clin. Pharmacol., 10: 77, 1990). Stimulants effective in the treatment of ADHD, d-amphetamine and methylphenidate, also are active on NE and DA systems. This suggests that the effects of the non-specific, partial agonist buspirone is through catecholaminergic mechanisms. Therefore, 5-HT_{1A} full agonists would not be predicted to alleviate inattention or hyperactivity.

### SUMMARY OF INVENTION

This invention relates to methods and compositions useful for treating ADHD in humans. The compounds for use in the invention are believed to be effective in the treatment of ADHD and to exhibit reduced side effects and are not expected to have abuse potential, as compared to other available therapeutics.

In one embodiment of this invention, a method of treating ADHD in humans is provided, which method comprises administering to an individual in need of treatment a therapeutically effective amount of one or more 5-HT_{1A} agonists, or pharmaceutically acceptable salts thereof.

Another embodiment of this invention is to provide the use of 5-HT_{1A} agonists, or pharmaceutically acceptable salts thereof, for the manufacture of a medicament for the treatment of ADHD.

The 5-HT_{1A} agonists of this invention may be full agonists or partial agonists, provided that they are effective in models of ADHD and/or the treatment of ADHD. Preferably, the 5-HT_{1A} agonists of this invention are selective for 5-HT_{1A} receptors over 5-HT_{1B/1D}, 5-HT₂, D₂, D₄, α₁, and α₂ receptors, and serotonin, dopamine and norephephrine transporters, especially over D₂ and α₁ receptors.

The 5-HT_{1A} agonists of this invention have intrinsic activity, as measured by maximal inhibition of forskolin-stimulated cAMP production as a proportion of the maximal effect produced by natural agonist 5-HT, that is 0.5-1.0. Preferably the intrinsic activity of the 5-HT_{1A} agonists of this invention is at least about 0.6-1.0. More preferably the intrinsic activity of the 5-HT_{1A} agonists of this invention is at least about 0.7-1.0. Most preferably the intrinsic activity of the 5-HT_{1A} agonists of this invention is at least about 0.8-1.0.

5-HT_{1A} receptor agonists that are useful in this invention include, but are not limited to, any one of, or any combination of the following compounds: flesinoxan [(+)(4-fluoro-N-[2-(4-[2-(hydroxymethyl)-1,4-benzodioxane-5-yl]1-piperazinyl)ethyl]benzamide) HCl], BAY x 3702 (R-(−)-2-[4-[(chroman-2-ylmethyl)-amino]-butyl]-1,1-dioxo-benzo[d]isothiazolone hydrochloride), F11440 [4-methyl-2-(4-[4-(pyrimidin-2-yl)-piperazino]-butyl)-2H,4H-1,2,4-triazin-3,5-dione], lesopitron (2-[4-[4-(4-chloro-1H-pyrazol-1-yl)-butyl]-1-piperazinyl]pyrimidine), LY228729 [(−)-4(dipropylamino)-1,3,4,5-tetrahydrobenz-[c,d,]indole-6-carboxamide]], (−) LY293284 [(−)-4R-6-acetyl-4-[di-n-propylamino)1,2,4,5-tetrahydrobenz-[c,d]indole], NAE-086 [(R)-3,4-dihydro-N-isopropyl-3-(N-isopropyl-N-propylamino)-2H-1-benzopyran-5-carboxamide], S14506 [1(2-[4-fluorobenzoylamino]ethyl)-4-(7-methoxynaphtyl)piperazine], S14671 [(4-[(thenoyl-2)aminoethyl]-1-(7-methoxynaphtylpiperazine], S16924 [(R)-2-([1-(2-[2,3-dihydrobenzo(1,4) dioxin-5-yloxy]-ethyl)-pyrrolidin-3yl])-1-(4-fluoro-phenyl)-ethanone], gepirone, and ipsapirone.

In an embodiment of this invention, the 5-HT_{1A} agonist is a compound of formula I: 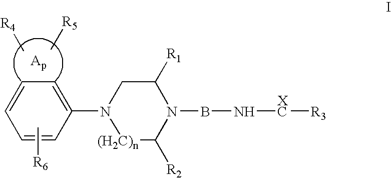
wherein:
R₁ and R₂ independently of each other represent hydrogen or an alkyl having 1-3 carbon atoms,
R₃ is hydrogen or straight or branched chain alkyl having 1-3 carbon atoms,
R₄ is hydrogen, halogen, alkyl having 1-3 carbon atoms, methylene, ethyldiene or vinyl, a straight or branched hydroxyalkyl group having 1-3 carbon atoms, which may be etherified or esterified, or an alkyl branched hydroxyalkyl group having 1-3 carbon atoms in the straight or branched alkyl group, an oxo group or a phenyl group,
R₅ is a hydrogen or fluoro atom,
n has the value 0 or 1,
A is the group —CH₂—CH₂— or —CH(CH₃)—CH₂—;
B is an aryl group or heteroaryl group which may be substituted with one or more substituents selected from the group consisting of halogen, trifluoromethyl, nitrile, nitro, alkoxy having 1-3 carbon atoms, hydroxy, esterified hydroxy, and alkyl having 1 or 2 carbon atoms; and
wherein
   the compound may be a racemate or a single diastereomer or enantiomer;
   or a pharmaceutically acceptable acid addition salt thereof.

In another embodiment of this invention, the 5-HT_{1A} agonist is compound of formula II: 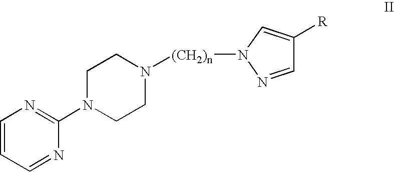
wherein:
n can have the value 1 to 6;
R is a hydrogen, a halogen, a lower alkyl radical having 1-4 carbon atoms, a heteroaryl radical, a sulpho radical, an N-substituted or N,N-disubstituted sulphamoyl radical, a nitro radical, a hydroxyl radical, an oxo radical, a lower alkoxyradical having 1-4 carbon atoms, a cyano radical, a lower alkylcarboxylate radical having 1-4 carbon atoms, an aryl or substituted aryl radical, or an amino or substituted amino radical of formula 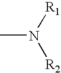
in which R₁ and R₂, independently are a hydrogen, an alkyl radical, an aryl radical, an alkylcarbonyl radical, an arylcarbonyl radical, an alkylsulphonyl radical or an arylsulphonyl radical, the alkyl fragments of these radicals containing from 1-4 carbon atoms; and
   wherein
the compound may be a racemate or a single diastereomer or enantiomer;
or a pharmaceutically acceptable acid addition salt thereof.

Another object of the invention is to provide pharmaceutical compositions for the treatment of ADHD that have reduced side effects as compared to other available treatments.

### BRIEF DESCRIPTION OF THE DRAWINGS

The following drawings are illustrative of embodiments of the invention and are not meant to limit the scope of the invention as encompassed by the claims.

FIGS. 1A-C—Graphs depict the relative response rate of C57BL/6J mice in the Peak Procedure (PIP 30 second reinforcement interval) after administration of 1, 2, or 4 mg/kg of d-amphetamine (triangles) compared to vehicle (circles). * p<0.05; ** p<0.01; *** p<0.001.

FIGS. 2A-B—Graphs depict the relative response rate of C3H mice in the Peak Procedure after administration of 0.03 mg/kg of flesinoxan (triangles) or vehicle (circles); 2A: PIP 30 second reinforcement interval; 2B: PIP 45 second reinforcement interval.

FIGS. 3A-B—Graphs depict the relative response rate of C3H mice in the Peak Procedure after administration of 0.1 mg/kg, of flesinoxan (triangles) or vehicle (circles); 3A: PIP 30 second reinforcement interval; 3B: PIP 45 second reinforcement interval.

FIGS. 4A-B—Graphs depict the relative response rate of C3H mice in the Peak Procedure after administration of 0.01 mg/kg (open circles) or 0.001 mg/kg (triangles) of 8-OH-DPAT or vehicle (circles); 4A: 30 second reinforcement interval; 4B: 45 second reinforcement interval.

FIG. 5—Graph depicts the effect of 4 mg/kg amphetamine on locomotor activity in coloboma mutant and wild-type mice (compared to vehicle), as measured by total distance traveled in a fixed time period. * p<0.05; ** p 0.01.

FIGS. 6A-F—Graphs depict the effect of 0.3 mg/kg flesinoxan (compared to vehicle) on locomotor activity in coloboma mutant (Cm) and wild-type (WT) mice; 6A,D total distance traveled in centimeters; 6B,E total distance traveled in centimeters per 5 minute block of the behavioral session; 6C,F: frequency of zone crossings per 5 minute block of the behavioral session.

FIGS. 7A-C—Graphs depict the effect of 0.1 mg/kg of 8-OH-DPAT on locomotor activity in coloboma mutant (Cm) and wild-type (WT) mice compared to saline vehicle; 7A: total distance traveled in centimeters; 7B: total distance traveled in centimeters per 5 minute block of the behavioral session; 7C: frequency of zone crossings per 5 minute block of the behavioral session.

### DETAILED DESCRIPTION OF THE INVENTION

This invention provides a method of treating ADHD in humans. As used herein, ADHD comprises the distinct sets of symptoms associated with the three subtypes defined in DSM-IV-TR, inattention, hyperactivity/impulsivity, or combined, which present in an individual as ADHD.

ADHD of the predominantly inattentive type is diagnosed if six (or more) of the following symptoms of inattention (and fewer than six of the hyperactivity-impulsivity symptoms below) have persisted for at least 6 months to a degree that is maladaptive and inconsistent with developmental level. The inattention component of ADHD may include one or more of the following symptoms: (a) often fails to give close attention to details or makes careless mistakes in schoolwork, work, or other activities, (b) often has difficulty sustaining attention in tasks or play activities, (c) often does not seem to listen when spoken to directly, (d) often does not follow through on instructions and fails to finish school work, chores, or duties in the workplace (not due to oppositional behavior or failure to understand instructions), (e) often has difficulty organizing tasks and activities, (f) often avoids, dislikes, or is reluctant to engage in tasks that require sustained mental effort (such as schoolwork or homework), (g) often loses things necessary for tasks or activities (e.g., toys, school assignments, pencils, books, or tools), (h) is often easily distracted by extraneous stimuli, and (i) is often forgetful in daily activities (DSM-IV-TR, supra).

ADHD of the predominantly hyperactive/impulsive type is diagnosed if six (or more) of the following symptoms of hyperactivity-impulsivity (and fewer than six of the inattention symptoms above) have persisted for at least 6 months to a degree that is maladaptive and inconsistent with developmental level. The hyperactivity component of ADHD may include one or more of the following symptoms: (a) often fidgets with hands or feet or squirms in seat, (b) often leaves seat in classroom or in other situations in which remaining seated is expected, (c) often runs about or climbs excessively in situations in which it is inappropriate (in adolescents or adults, may be limited to subjective feelings of restlessness), (d) often has difficulty playing or engaging in leisure activities quietly, (e) is often “on the go” or often acts as if “driven by a motor,” and (f) often talks excessively. The impulsivity component of ADHD may include one or more of the following symptoms: (g) often blurts out answers before questions have been completed, (h) often has difficulty awaiting turn, and (i) often interrupts or intrudes on others (e.g. butts into conversations or games) (DSM-IV-TR, supra).

The most common subtype of ADHD is the combined type, which comprises all three sets of symptoms, inattention, hyperactivity and impulsivity. Combined-type ADHD is diagnosed if six (or more) symptoms of inattention and six (or more) symptoms of hyperactivity/impulsivity have persisted for at least 6 months (DSM-IV-TR, supra).

ADHD of the combined type, as well as the inattentive and hyperactive/impulsive subtypes, may be treated according to this invention. Other forms of ADHD, to the extent that they are clinically distinct from that described in DSM-IV-TR, are also within the scope of this invention.

Unlike traditional therapeutics, which have the potential to be abused and/or have undesirable side effects, the present invention is not expected to have the abuse potential of psychostimulants, the most widely prescribed current pharmacological treatment, and may have a side effect profile distinct from other types of pharmacologic therapeutics. Therefore, an advantage of the method of ADHD treatment provided by this invention is that certain of the undesirable side effects may be reduced or avoided.

As discussed above, ADHD is diagnosed based on an individual possessing symptoms in the symptom clusters inattentiveness, hyperactivity and impulsiveness, those terms are clinically recognized in the art, as for example, DSM-IV-TR.

Preferably, ADHD is treated according to this invention by administering therapeutic amounts of compounds that are selective 5-HT_{1A} agonists. “Selective.” as used herein, means having a greater affinity for 5-HT_{1A} receptors than for 5-HT_{1B/1D}, 5-HT₁, D₂, D₄, α₁, or α₂ receptors and for serotonin transporter (SERT), dopamine transporter (DAT), and norepinephrine transporter (NET). Selectivity may be based on relative Kᵢ values or on relative affinity constants determined using saturation binding studies coupled with Scatchard analysis to determine K_{d} values.

Preferably, the 5-HT_{1A} agonists of this invention have an affinity for 5-HT_{1A} receptors that differs from 5-HT_{1B/1D}, 5-HT₂, D₂, D₄, α₁ or α₂ receptors or SERT, DAT, or NET, by at least 1 pKᵢ (e.g., 1 order of magnitude). More preferably, the 5-HT_{1A} agonists of this invention have an affinity for 5-HT_{1A} receptors that differs from that for D₂ receptors by at least about 2 pKᵢ (e.g., 2 order of magnitude). Most preferably, the 5-HT_{1A} agonists of this invention have an affinity for 5-HT_{1A} receptors that differs from their affinities for 5-HT_{1B/1D}, 5-HT₂, D₂, D₄, α₁ or α₂ receptors or SERT, DAT, or NET by at least about 2 pKᵢ (e.g., 2 order of magnitude).

As used herein, pK₁ means the negative log of the affinity constant (Kᵢ) expressed in M. For example, flesinoxan has an affinity for 5-HT_{1A} of Kᵢ=1.7 nM (1.7×10⁻⁹M) which equals 8.77 pKᵢ, and an affinity for D₂ receptors of Kᵢ=140 nM (1.4×10⁻⁷M), which equals 6.85 pKᵢ (see Schipper et al., 1991, supra). Accordingly, for flesinoxan, the difference in affinity for 5-HT_{1A} and D₂ receptors (ΔpKᵢ) is about 1.92. Based on the Kᵢ values reported by Schipper et al., 1991, supra, the ΔpKᵢ for 5-HT_{1A} and D₂ receptors is 2.94 for 8-OH-DPAT and is 0.44 for buspirone. Koek et al., J. Pharmacol. Exp. Ther., 287:266-283, 1998, have reported comparable pKᵢ values for 5-HT_{1A} and D₂ receptors for flesinoxan: 8.91 and 7.05, respectively (ΔpKᵢ=1.86), and for buspirone: 7.5 and 7.43, respectively (ΔpKᵢ=0.07). For another selective 5-HT_{1A} agonist F11440, Koek et al., 1998, supra, report a pKᵢ of 8.33 and 5.75 for 5-HT_{1A} and D₂ receptors, respectively (ΔpKᵢ=2.58). Thus, a 5-HT_{1A} agonist that is reported to have significant (or equal) agonist activity at D₂ receptors, such as sunipetron (U.S. Pat. No. 6,300,329), or affinity for D₂ receptors that is functionally equivalent to 5-HT_{1A} receptors, such as buspirone (ΔpKᵢ<1), is not within the scope of this invention.

Flesinoxan (+)(4-fluoro-N-[2-[4-[2-(hydroxymethyl)-1,4-benzodioxane-5-yl]1-piperazinyl]ethyl]benzamide hydrochloride), then, is a selective 5-HT_{1A} agonist. Other examples of selective 5-HT_{1A} agonists include BAY x 3702 (R-(−)-2-[4-[(chroman-2-ylmethyl)-amino]-butyl]-1,1-dioxo-benzo[d]isothiazolone hydrochloride), F11440 [4-methyl-2-(4-[4-(pyrimidin-2-yl)-piperazino]-butyl)-2H,4H-1,2,4-triazin-3,5-dione], and ipsapirone.

Preferably, the 5-HT_{1A} agonists for use in this invention have an intrinsic activity at 5-HT_{1A} receptors greater than 0.5. “Intrinsic activity,” is the proportion of maximal inhibition of forskolin-stimulated cAMP achieved by a test compound relative to the maximal inhibition of forskolin-stimulated cAMP achieved by the natural agonist 5-HT (see Koek et al., 1998, supra). Inhibition of forskolin-stimulated cAMP production may be measured in stably transfected cell lines (for example, HeLa or CHO cells) that express 5-HT_{1A} receptors (Pauwels, et al. 1993, supra; Koek et al., 1998, supra; Evans et al., J. Pharmacol. Exp. Ther., 297(3):1025-35, 2001). For use in this invention, 5-HT_{1A} agonists display intrinsic activity at 5-HT_{1A} receptors of at least 0.5-1.0. Preferably, the 5-HT_{1A} agonists of this invention have an intrinsic activity of at least about 0.6-1.0. Even more preferably, the 5-HT_{1A} agonists of this invention have an intrinsic activity of at least about 0.7-1.0. Most preferably, the 5-HT_{1A} agonists of this invention have an intrinsic activity of at least about 0.8-1.0 (see Koek et al., 1998, supra).

The azapirone derivatives gepirone and ipsapirone are 5-HT_{1A} partial agonists chemically related to buspirone that have significant affinity for D₂ receptors as well as 5-HT_{1A} receptors. Nevertheless, gepirone has sufficient intrinsic activity at the 5-HT_{1A} receptor (0.77, Koek et al., 1998, supra), that it also is expected to be a useful 5-HT_{1A} agonist in this invention.

The following compounds have the preferred intrinsic activity: flesinoxan (0.93), F11440 (1.0), LY228729 (0.88), S14506 (0.95), lesopitron (0.70), and gepirone (0.77).

The compounds having formulas I or II below are preferred for use with this invention.

Treatment of ADHD according to this invention is provided by administering to an individual in need of treatment a therapeutically effective amount of a compound of formula I: 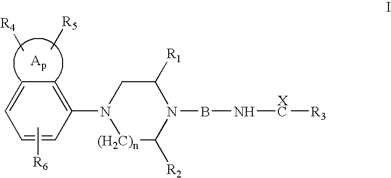
wherein:
R₁ and R₂ independently of each other represent hydrogen or an alkyl having 1-3 carbon atoms;
R₃ is an aryl group or heteroaryl group which may be substituted with one or more substituents selected from the group consisting of halogen, trifluoromethyl, nitrile, nitro, alkoxy having 1-3 carbon atoms, hydroxy, esterified hydroxy, and alkyl having 1 or 2 carbon atoms;
X is O, S, or NH;
B is the group —CH₂—CH₂— or —CH(CH₃)—CH₂—;
n has the value 0 or 1;
p has the value 0 or 1;
   where p has the value 1,
      A is O—CH₃, or forms, with the two carbon atoms of the phenyl group, an optionally substituted, entirely or partly unsaturated, cyclic group having 5-7 atoms in the ring, which comprises 1-3 hetero atoms from the group O, S, and N, with the proviso that the sum of the number of oxygen and sulfur atoms is at most two,
         and %% here A is not O—CH₃,
         R₄ is hydrogen or straight or branched chain alkyl having 1-3 carbon atoms, and
         R₅ is hydrogen, halogen, alkyl having 1-3 carbon atoms, methylene, ethyldiene or vinyl, a straight or branched hydroxyalkyl group having 1-3 carbon atoms, which may be etherified or esterified, or an alkyl branched hydroxyalkyl group having 1-3 carbon atoms in the straight or branched alkyl group, an oxo group or a phenyl group; and
R₆ is a hydrogen or fluoro atom.
wherein
the compound may be a racemate or a single diastereomer or enantiomer;
or a pharmaceutically acceptable acid salt thereof.

In a preferred embodiment of this invention, the 5-HT_{1A} agonist is a compound of formula I, wherein
R₁, R₂, and R₆ are hydrogen;
R₃ is a lipophilic aromatic alkyl, selected from the group consisting of benzene, halogenated benzene, cyclohexane, and 2-thiophene;
X is O, S, or NH;
B is the group —CH₂—CH₂—;
n has the value 1;
p has the value 0 or 1;
   where p is 1,
      A is O—CH₃, or forms, with the two carbon atoms of the phenyl group, an optionally substituted benzodioxane, a hydroxyalkyl having 1-2 carbon atoms, or a furan,
         and where A is not O—CH₃,
         R₄ is H, and R₅ is H, or chiral —CH₂OH— at the 2 position of the benzodioxane ring;
            or pharmaceutically acceptable salts thereof, which is administered to individuals to provide treatment of ADHD.

In a more preferred embodiment of this invention, the 5-HT_{1A} agonist if formula I is flesinoxan [(+)(4-fluoro-N-[2-[4-[2-(hydroxymethyl)-1,4-benzodioxane-5-yl] 1-piperazinyl]ethyl]benzamide)], or pharmaceutically acceptable salts thereof, preferably hydrochloride, wherein R₁, R₂, and R₆ are hydrogen; R₃ is a halogenated benzene group, having a fluoro in the para position; X is O; n has the value 1; p has the value 1; A is benzodioxane; R₄ is hydrogen; R₅ is chiral —CH₂OH— at the 2 position of the benzodioxane ring; and B is the group —CH₂—CH₂—; and which is administered to individuals to provide treatment of ADHD.

The compounds of formula I described above, and the preferred and more preferred embodiments, and their method of synthesis are described in U.S. Pat. No. 4,833,142; U.S. Pat. No. 5,914,263; and European Patent No. 138,280; and Kuipers et al., J. Med. Chem. 40:300-312, 1997; which are incorporated herein by reference in their entireties. The affinity of phenyl piperazines for 5-HT_{1A} receptors is described by Schipper et al., 1991, supra; and Kuipers et al., 1997, supra; which are incorporated herein by reference in their entireties.

In still another aspect of this invention, the 5-HT_{1A} agonist is a compound of the formula II: 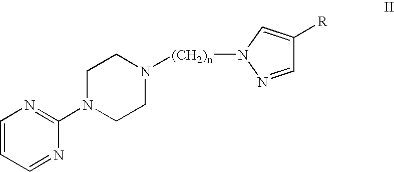
wherein:
n can have the value 1 to 6;
R is a hydrogen, a halogen, a lower alkyl radical having 1-4 carbon atoms, a heteroaryl radical, a sulpho radical, an N-substituted or N,N-di-substituted sulphamoyl radical, a nitro radical, a hydroxyl radical, an oxo radical, a lower alkoxyradical having 1-4 carbon atoms, a cyano radical, a lower alkylcarboxylate radical having 1-4 carbon atoms, an aryl or substituted aryl radical, or an amino or substituted amino radical of formula 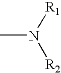
in which R₁ and R₂, independently are a hydrogen, an alkyl radical, an aryl radical, an alkylcarbonyl radical, an arylcarbonyl radical, an alkylsulphonyl radical or an arylsulphonyl radical, the alkyl fragments of these radicals containing from 1-4 carbon atoms; and
   wherein
the compound may be a racemate or a single diastereomer or enantiomer;
or a pharmaceutically acceptable acid addition salt thereof.

A preferred compound of formula II of this invention is lesopitron (2-[4-[4-(4-chloro-1H-pyrazol-1-yl)-butyl]-1-piperazinyl] pyrimidine), or a pharmaceutically acceptable salt thereof, preferably dihydrochloride, wherein n has the value 4; and R is chloro; which is administered to individuals to provide treatment of ADHD.

The compounds of formula II, described above, including lesopitron, and their method of synthesis are described in U.S. Pat. Nos. 5,128,343; 5,182,281; 5,162,323; 5,536,836; and 5,872,125; and EP 382,637 B1; which are incorporated herein by reference in their entireties. The affinity of lesopitron for 5-HT_{1A} receptors is described by Costall et al., J. Pharmacol. Exp. Ther., 262:90-98, 1992; and Farré, Behav. Pharmacol., 3(Suppl.1):23, 1992. Lesopitron is reported to have virtually no affinity for other 5-HT receptor subtypes or other neurotransmitter receptors.

ADHD is treated according to this invention by administering therapeutic amounts of compounds according to formulas I or II, or combinations thereof.

This invention also includes the use of prodrugs of the compounds of formulas I and II, specifically derivatives of the compounds of formulas I and II that are inactive but are converted to an active form in the body following administration.

ADHD is also treated according to this invention by administering therapeutic amounts of other 5-HT_{1A} agonists. A non-exhaustive list of other 5-HT_{1A} agonists that would be useful in this invention includes, but is not limited to, the following: BAY x 3702 (R-(−)-2-[4-[(chroman-2-ylmethyl)-amino]-butyl]-1,1-dioxo-benzo[d]isothiazolone hydrochloride), F11440 [4-methyl-2-(4-[4-(pyrimidin-2-yl)-piperazino]-butyl)-2H,4H-1,2,4-triazin-3,5-dione], LY228729 [(−)-4(dipropylamino)-1,3,4,5-tetrahydrobenz-[c,d,]indole-6-carboxamide]], (−) LY293284 [(−)-4R-6-acetyl-4-[di-n-propylamino)1,2,4,5-tetrahydrobenz-[c,d]indole], NAE-086 [(R)-3,4-dihydro-N-isopropyl-3-(N-isopropyl-N-propylamino)-2H-1-benzopyran-5-carboxamide], S14506 [1 (2-[4-fluorobenzoylamino]ethyl)-4-(7-methoxynaphtyl)piperazine], S14671 [(4-[(thenoyl-2)aminoethyl]-1-(7-methoxynaphtylpiperazine], and S16924 [(R)-2-([1-(2-[2,3-dihydrobenzo(1,4) dioxin-5-yloxy]-ethyl)-pyrrolidin-3yl])-1-(4-fluoro-phenyl)-ethanone]. Preferred compounds from this group for use in this invention are BAY x 3702, F11440, LY228729, (−) LY293284, and S14506. These 5-HT_{1A} agonists are described in the following articles, which are incorporated herein by reference in their entireties: Koek et al., 1998, supra; Foreman, et al., J. Pharmacol. Exp. Ther., 267:58-71, 1993; Foreman et al. J. Pharmacol. Exp. Ther. 270(3):1270-81, 1994; Gobert et al., J. Pharmacol. Exp. Ther., 273:1032-46, 1995; Millan et al., J Pharmacol. Exp. Ther., 262:451-63; Colpaert et al., Drug Devel. Res. 26:4148, 1992; Rënyi ct al., J. Pharmacol. Exp. Ther., 299:883-893, 2001; DeVry et al., J. Pharmacol. Exp. Ther., 284(3):1082-94, 1998.

5-HT_{1A} agonists for use in this invention may be identified by a number of assays, known in the art that measure receptor affinity or functional parameters (including intrinsic activity) described above. These assays include, but are not limited to (I) in vitro affinity binding assays, for example in tissue or cell preparations, and (2) functional assays.

Affinity of a particular compound of the invention at 5-HT_{1A} receptors can be determined, for example, using a single saturating concentration according to any of the procedures well known in the art, using [³H]-8-OH-DPAT as ligand for displacement, in membrane preparations from brain tissue or transfected cells stably expressing 5-HT_{1A} receptors. Determination of a lesser affinity of the compounds of this invention for other receptors may be determined by methods known in the art. An exemplary protocol for a 5-HT_{1A} binding assay is described briefly below. To provide means to assess the neurotransmitter receptor selectivity of a target 5-HT_{1A} agonist, exemplary ligand binding assay protocols for key neurotransmitter receptors, 5-HT_{1A/1D}, 5-HT₂, D₂, D₄, α₁, and α₂, are described briefly below, as are binding assay protocols for serotonin transporter (SERT), dopamine transporter (DAT) and norepinephrine transporter (NET). Alternative ligand binding assay protocols for these receptors, as well as ligand binding assay protocols for other neurotransmitter receptors, may be found in the art.

5-HT_{1A} receptor binding assays may be carried out, for example, in HEK-293 cells expressing human recombinant 5-HT_{1A} receptors, using a final concentration of [³H]8-OH-DPAT (221 Ci/mmol) of 0.5 nM. The reference compound also is 8-OH-DPAT; Kᵢ of reference compound 8-OH-DPAT in this assay is approximately 1.6 nM. Reactions are carried out in 50 mM TRIS-HCl (pH 7.4) containing 10 mM MgCl₂, 0.5 mM EDTA and 0.1% ascorbic acid for 60 minutes at 25° C. After termination of the reaction by rapid vacuum filtration onto glass fiber filters, radioactivity trapped onto the filters is determined and compared to control values in order to ascertain any binding of test compound(s) to 5-HT_{1A} binding sites. The sensitivity of the assay is approximately K_{D}=0.8 nM and Bₘₐₓ=622 pmol/mg protein. For reference, see Hoyer et al., Eur. J. Pharmacol., 118:13-23, 19S5; Schoeffter and Hoyer, Naunyn-Schmiedeberg's Arch. Pharmacol., 340:135-38, 1989, which are incorporated herein by reference, in their entireties.

5-HT_{1B/1D} receptor binding assays may be carried out, for example, in membrane preparations from rat or bovine striatum or human cerebral cortex, using a final concentration of [³H]5-carboxyamidotryptamine (5-CT) (20-70 Ci/mmol) of 0.75 nM or 2 nM in cortex. The reference compound is 5-CT; Kᵢ of reference compound 5-CT in this assay is approximately 0.7-1.1 nM. Alternatively, one may use a final concentration of [¹²⁵I]iodocyanopindolol (ICP) (2200 Ci/mmol) of 0.15 nM in striatum, and 5-HT as the reference compound; Kᵢ of reference compound in this assay 5-HT is approximately 13.8 nM. For the assay using 5-CT (primarily 5-HT_{1B}), reactions are carried out in 50 mM TRIS-HCl (pH 7.7) containing 4 mM CaCl₂, 100 nM 8-OH-DPAT, 100 nM mesulergine, 10 μm pargyline, and 0.1% ascorbic acid for 60 minutes at 25° C. For the assay using ICP (primarily 5 HT_{1D})), reactions are carried out in 50 mM TRIS-HCl (pH 7.4) containing 60 μM (−)isoproterenol for 60 minutes at 37° C. Reactions are terminated by rapid vacuum filtration onto glass fiber filters, and radioactivity trapped onto the filters is determined and compared to control values in order to ascertain any binding of test compound(s) to 5-HT_{1B/1D} binding sites. The sensitivity of the assay is approximately K_{D}=0.12-1.0 nM and Bₘₐₓ=2.1-60 fmol/mg tissue. For reference, see Hoyer et al., 1985, supra; Schoeffter and Hoyer, 1989, supra; Waeber et al., Naunyn-Schmiedeberg's Arch. Pharmacol., 337:595-601, 1988, which are incorporated herein by reference, in their entireties.

5-HT₂ (5-HT_{2A}) receptor binding assays may be carried out, for example, in membrane preparations from rat or human cerebral cortex, using a final concentration of [³H]ketanserin (60-90 Ci/mmol) of 1.0-2.0 nM. The reference compound may be methysergide or ketanserin. Kᵢ of reference compound methysergide in this assay is approximately 1.6 nM; Kᵢ of reference compound ketanserin in this assay is approximately 4.0 nM. Reactions are carried out in 50 mM TRIS-HCl (pH 7.6) for 60 minutes at 37° C. or for 90 minutes at 25° C. After termination of the reaction by rapid vacuum filtration onto glass fiber filters, radioactivity trapped onto the filters is determined and compared to control values in order to ascertain any binding of test compound(s) to 5-HT₂ binding sites. The sensitivity of the assay is approximately K_{D}=0.43-2.0 nM and Bₘₐₓ=10.0-30.9 fmol/mg protein. For reference, see Leysen et al. Mol. Pharmacol., 21:301-14, 1982; Martin and Humphrey, Neuropharmacol., 33(3/4):261-73, which are incorporated herein by reference, in their entireties.

D₂ receptor binding assays may be carried out, for example, in CHO cells expressing human recombinant D₂ receptors, using a final concentration of [³H]spiperone (20-60 Ci/mmol) of 0.2 nM. The reference compound is haloperidol; Kᵢ of reference compound haloperidol in this assay is approximately 2.8 nM. Reactions are carried out in 50 mM TRIS-HCl (pH 7.4) containing 120 mM NaCl, 5 mM KCl, 5 mM MgCl₂, 1 mM EDTA for 60 minutes at 25° C. After termination of the reaction by rapid vacuum filtration onto glass fiber filters, radioactivity trapped onto the filters is determined and compared to control values in order to ascertain any binding of test compound(s) to D₂ binding sites. The sensitivity of the assay is approximately K_{D}=0.1 nM and Bₘₐₓ=1.5 pmol/mg protein. For reference, see Jarvis et al. J. Receptor Res. 13(10-4):573-590, 1993; Gundlach et al., Life Sciences, 35:1981-88, 1984, which are incorporated herein by reference, in their entireties.

D₄ receptor binding assays may be carried out, for example, in CHO cells expressing human recombinant D₄ receptors, using a final concentration of [³H]YM-09151-2 (70-87 Ci/mmol) of 0.3 nM. The reference compound is haloperidol; Kᵢ of reference compound haloperidol is approximately 0.8 nM. Reactions are carried out in 50 mM TRIS-HCl (pH 7.4) containing 5 mM KCl, 5 mM MgCl₂, 5 mM EDTA, and 1.5 mM CaCl₂, for 60 minutes at 22° C. After termination of the reaction by rapid vacuum filtration onto glass fiber filters, radioactivity trapped onto the filters is determined and compared to control values in order to ascertain any binding of test compound(s) to D₄ binding sites. The sensitivity of the assay is approximately K_{D}=0.26 nM and Bₘₐₓ=43 pmol/mg protein. For reference, see Van Tol, et al., Nature, 350:610, 1991; Van Tol, et al., Nature, 358:149, 1992; Seeman et al., Eur. J. Pharmacol., 233:173, 1993, which are incorporated herein by reference, in their entireties.

α₁ receptor binding assays may be carried out, for example, in rat forebrain membranes, using a final concentration of[³H]7-MeOxy-prazosin (70-87 Ci/mmol) of 0.3 nM. The reference compound is phentolamine mesylate; Kᵢ of reference compound phentolamine mesylate in this assay is approximately 5.4 nM. Reactions are carried out in 50 mM TRIS-HCl (pH 7.7) for 60 minutes at 25° C. After termination of the reaction by rapid vacuum filtration onto glass fiber filters, radioactivity trapped onto the filters is determined and compared to control values in order to ascertain any binding of test compound(s) to α₁ binding sites. The sensitivity of the assay is approximately K_{D}=0.2 nM and Bₘₐₓ=95 fmol/mg protein. For reference, see Timmermans, et al. Mol. Pharmacol. 20: 295-301, 1981, with modifications; and Reader, et al. J. Neutral Transmission. 68: 79-95, 1987, which are incorporated herein by reference, in their entireties.

α₂ receptor binding assays may be carried out, for example, in rat cortical membranes, using a final concentration of [³H]RX 821002 (40-60 Ci/mmol) of 1.0 nM. The reference compound is phentolamine mesylate; Kᵢ of reference compound phentolamine mesylate in this assay is approximately 3.2 nM. Reactions are carried out in 50 mM TRIS-HCl (pH 7.4) for 75 minutes at 25° C. After termination of the reaction by rapid vacuum filtration onto glass fiber filters, radioactivity trapped onto the filters is determined and compared to control values in order to ascertain any binding of test compound(s) to α₂ binding sites. The sensitivity of the assay is approximately K_{D}=1.5 nM and Bₘₐₓ=60 fmol/mg protein. For reference, see O'Rourke, et al. J. Pharmacol. Exp. Ther. 268(3): 1362, 1993; and Reader, et al. J. Neural Transmission. 68: 79-95, 1987, which are incorporated herein by reference, in their entireties.

SERT binding assays may be carried out, for example, in human platelet membranes, using a final concentration of [³H] citalopram, N-Methyl (70-87 Ci/mmol) of 0.7 nM. The reference compound is imipramine; Kᵢ of reference compound imipramine in this assay is approximately 4.0 nM. Reactions are carried out in 50 mM TRIS-HCl (pH 7.4), containing 120 mM NaCl and 5 mM KCl for 60 minutes at 25° C. After termination of the reaction by rapid vacuum filtration onto glass fiber filters, radioactivity trapped onto the filters is determined and compared to control values in order to ascertain any binding of test compound(s) to SERT binding sites. The sensitivity of the assay is approximately K_{D}=2.5 nM and Bₘₐₓ=425 fmol/mg protein. For reference, see D'Amato, et al. J. Pharmacol. & Exp. Ther. 242: 364-371, 1987; and Brown, et al. Eur. J. Pharmac. 123: 161-165, 1986, which are incorporated herein by reference, in their entireties.

DAT binding assays may be carried out, for example, in guinea pig striatal membranes, using a final concentration of [³H]WrN,35,428 (60-87 Ci/mmol) of 2.0 nM. The reference compound is GBR-12909; Kᵢ of reference compound GBR-12909 in this assay is approximately 7.1 nM. Reactions are carried out in. 50 mM TRIS-HCl (pH 7.4) containing 120 mM NaCl for 2 hours at 0-4° C. After termination of the reaction by rapid vacuum filtration onto glass fiber filters, radioactivity trapped onto the filters is determined and compared to control values in order to ascertain any binding of test compound(s) to DAT binding sites. The sensitivity of the assay is approximately K_{D}=28.0 nM and Bₘₐₓ=13 fmol/mg protein. For reference, see Madras, et al. Mol. Pharmacol. 36: 518-524, 1989; and Javitch, et al. Mol. Pharmacol. 26: 35-44, 1984, which are incorporated herein by reference, in their entireties.

NET binding assays may be carried out, for example, in rat forebrain membranes, using a final concentration of [³H]nisoxetine (60-85 Ci/mmol) of 1.0 nM. The reference compound is desipramine; Kᵢ of reference compound desipramine in this assay is approximately 0.7 nM. Reactions are carried out in 50 mM TRIS-HCl (pH 7.4), containing 300 mM NaCl and 5 mM KCl for 4 hours at 0°-4° C. After termination of the reaction by rapid vacuum filtration onto glass fiber filters, radioactivity trapped onto the filters is determined and compared to control values in order to ascertain any binding of test compound(s) to NET binding sites. The sensitivity of the assay is approximately K_{D}=0.8 nM and Bₘₐₓ=10.5 fmol/mg protein. For reference, see Raisman, et al. Eur. J. Pharmacol. 78: 345-351, 1982; and Langer, et al. Eur. J. Pharmac. 72: 423, 1981, which are incorporated herein by reference, in their entireties.

Functionally, 5-HT_{1A} agonists and partial agonists have been shown to inhibit forskolin-induced cAMP production in HeLa or CHO cells that are stably transfected to express 5-HT_{1A} receptors, as described by Pauwels et al., Biochem. Pharmacol. 45:375-383, 1993; Koek et al., 1998 supra; and Evans et al., 2001, supra; which are incorporated by reference herein, in their entireties. 5-HT_{1A} agonists also induce hypothermia and spontaneous tail flicks in rodents, as described by Hjorth et al., 1985, supra; Millan et al., Eur. J. Pharmacol. 203:319-22, 1991; Millan et al., J. Pharmacol. Exp. Ther. 256:973-82, 1991, which are incorporated by reference herein, in their entireties.

5-HT_{1A} agonists for use in this invention are expected to display positive results in models of ADHD, such as improved performance in the peak procedure and reduced locomotor activity in spontaneously hyperactive animals, as described herein, as well as in clinical studies of ADHD patients.

The utility of the compounds of this invention for treating ADHD is based on the surprising discovery disclosed herein that flesinoxan and 8-OH-DPAT share certain activity profiles with other compounds known to be useful for treating such conditions. Amphetamines enhance monoaminergic transmission; however, their mechanism of action in ADHD is still the subject of much speculation. Without being bound by theory, one possible mechanism is the enhancement of dopamine release in those areas of the brain that are involved in attentional mechanisms, such as the frontal cortex, however, such a model seems to be overly simplistic and incomplete (Nestler, Hyman, & Malenka, “Sleep, Arousal and Attention,” Ch. 18, In: Molecular Neuropharmacology: A Foundation for Clinical Neuroscience, McGraw Hill, 2001). Psychoactive substances such as amphetamines typically show a U-shape curve, with low doses being effective, and high doses being disruptive.

The mechanism underlying these U-shape curves is poorly understood, with one possibility being the differential action on pre- and postsynaptic dopamine D2 receptors. It is possible that low doses preferentially affect the post- (or pre-) synaptic receptors, and that only higher doses affect both types. The differential action could be the result of different binding characteristics (due to subtle changes in the receptors), or to differences in the amount of receptor reserve (where high receptor reserve results in a stronger effect). This dual pre- and postsynaptic action of dopamine (and of dopamine agonists) is mimicked in the serotonergic system, in which the 5-HT_{1A} and 5-HT_{1B} receptors exist as both autoreceptors (presynaptic) and heteroreceptors (postsynaptic) and have opposite effects. Presynaptic action typically results in a reduction of neurotransmitter release (and less activation of target receptors), whereas postsynaptic action results in enhanced activation of target receptors.

Although the main target of amphetamine-like drugs (and of bupropion, one antidepressant used for ADHD when adverse reactions prevent the use of psychostimulants) is the dopaminergic system, strong interactions between dopamine and serotonin are known. As a result, drugs that affect the serotonin system will very likely have secondary effects in the dopaminergic system. Moreover, serotonergic drugs that have a dual pre- and postsynaptic action would be expected to show U-shaped responses. Thus, a drug that has positive effects on performance at low doses, and disrupts performance at high doses, may be a drug that mimics amphetamine-like effects, and therefore may be of value in the treatment of ADHD.

The dose of the compound used in treating ADHD in accordance with this invention will vary in the usual way with the seriousness of the disorder, the weight, and metabolic health of the individual in need of treatment. The preferred initial dose for the general patient population will be determined by routine dose-ranging studies, as are conducted, for example, during clinical trials. Therapeutically effective doses for individual patients may be determined, by titrating the amount of drug given to the individual to arrive at the desired therapeutic or prophylactic effect, while minimizing side effects. A preferred initial dose for flesinoxan is between about 0.04 mg/day and 4 mg/day, in single or multiple daily doses. A more preferred initial dose for flesinoxan is between about 0.1 mg/day and 1 mg/day. A most preferred initial dose for flesinoxan between about 0.1 mg/day and 0.5 mg/day. For the other 5-HT_{1A} agonists of this invention, a preferred initial dose is between about 0.01 mg/day and 100 mg/day, in single or multiple daily doses. A more preferred initial dose for the other 5-HT_{1A} agonists of this invention is between about 0.1 mg/day and 10 mg/day. A most preferred initial dose for the other 5-HT_{1A} agonists of this invention is between about 0.1 mg/day and 2 mg/day.

Administration of the compounds of this invention may be by any method used for administering therapeutics, such as for example oral, parenteral, intravenous, intramuscular, subcutaneous, or rectal administration.

In addition to comprising the therapeutic compounds for use in this invention, the pharmaceutical compositions for use with this invention may also comprise a pharmaceutically acceptable carrier. Such carriers may comprise additives, such as preservatives, excipients, fillers, wetting agents, binders, disintegrants, buffers may also be present in the compositions of the invention. Suitable additives may be, for example magnesium and calcium carbonates, carboxymethylcellulose, starches, sugars, gums, magnesium or calcium stearate, coloring or flavoring agents, and the like. There exists a wide variety of pharmaceutically acceptable additives for pharmaceutical dosage forms, and selection of appropriate additives is a routine matter for those skilled in art of pharmaceutical formulation.

The compositions may be in the form of tablets, capsules, powders, granules, lozenges, suppositories, transdermal delivery devices, aerosols, pumps, reconstitutable powders, or liquid preparations such as oral or sterile parenteral solutions or suspensions.

In order to obtain consistency of administration it is preferred that a composition of the invention is in the form of a unit dose. Unit dose forms for oral administration may be tablets, capsules, and the like, and may contain conventional excipients such as binding agents, for example syrup, acacia, gelatin, sorbitol, tragacanth, or polyvinylpyrrolidone; and carriers or fillers, for example lactose, sugar, maize-starch, calcium phosphate, sorbitol or glycine. Additives may include disintegrants, for example starch, polyvinylpyrrolidone, sodium starch glycolate or microcrystalline cellulose; preservatives, and pharmaceutically acceptable wetting agents such as sodium lauryl sulphate.

In addition to unit dose forms, multi-dosage forms are also contemplated to be within the scope of the invention. Delayed-release compositions, for example those prepared by employing slow-release coatings, micro-encapsulation, and/or slowly-dissolving polymer carriers, will also be apparent to those skilled in the art, and are contemplated to be within the scope of the invention. Delayed release compositions are especially desirable for transdermal delivery devices.

The solid oral compositions may be prepared by conventional methods of blending, filling, tabletting or the like. Repeated blending operations may be used to distribute the active agent throughout those compositions employing large quantities of fillers. Such operations are conventional in the art. The tablets may be coated according to methods well known in normal pharmaceutical practice, for example with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol syrup, methyl cellulose, gelatin, hydroxyethylcellulose, carboxymethylcellulose, aluminum stearate gel, and hydrogenated edible fats; emulsifying agents, for example lecithin, sorbitan monooleate, or acacia; non-aqueous vehicles (which may include edible oils), for example almond oil or fractionated coconut oil, oily esters such as esters of glycerine, propylene glycol, or ethyl alcohol; preservatives, for example methyl or propyl p-hydroxybenzoate or sorbic acid; and if desired conventional flavoring or coloring agents.

For transdermal administration, the compound may be dispersed within a physiologically compatible matrix or carrier, which is then provided in the form of an ointment, gel, cream, lotion, or other components of topical compositions that are known to the art. Preferably transdermal administration is via a skin patch or other known transdermal delivery device which contains a saturated or unsaturated formulation. The formulation may optionally include permeation enhancers, and an anti-irritant, where indicated. The matrix or carrier may also contain excipients, inert fillers, dyes, pigments, and other conventional components of pharmaceutical products or transdermal devices known to the art, for example, hydrophilic water absorbing polymers such as polyvinyl alcohol and polyvinyl pyrrolidone individually or in combination.

For parenteral administration, fluid unit dosage forms are prepared utilizing the compound and a sterile vehicle, and, depending on the concentration used, can be either suspended or dissolved in the vehicle. In preparing solutions the compound can be dissolved in water or saline for injection and filter sterilized before filling into a suitable vial or ampoule and sealing. Advantageously, additives such as a local anaesthetic, preservative and buffering agent can be dissolved in the vehicle. Suitable buffering agents are for example, phosphate and citrate salts. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by conventional means, for example by exposure to radiation or ethylene oxide, before being suspended in the sterile vehicle. Advantageously, a surfactant or wetting agent is included in the composition to facilitate uniform distribution of the compound.

The invention will be explained in more detail below by way of examples, which illustrate the effectiveness of prototypical compounds flesinoxan and 8-OH-DPAT in treating ADHD.

### EXAMPLE 1

The peak procedure is a behavioral model designed to assess an animal's ability to learn an appropriate time period in which to perform a task and a time period in which the animal will be rewarded if the task is performed. The model provides information concerning excitatory and inhibitory components of behavior, as subjects must respond to perform a task when appropriate and stop responding in an “empty trial” when time for reward has elapsed and the reward has not been delivered. The task is sensitive to conditions where there is a failure in inhibitory mechanisms, such as seems to be the case for ADHD (Pliszka et al., Biol. Psychiatry, 48:238-46, 2000).

In the peak procedure, mice are trained to work for food that is delivered at the same time in each trial, but withdrawn in some unreinforced trials. Typically, the response rate increases up to a maximum around the reinforcement time, and then decreases to a low toward the end of the trial. The shape of the response rate indicates whether the animal is sensitive to the time of reinforcement. To be able to perform well in this model, the animals need to be able to learn several tasks. First, the animal must make an association between a response (lever pressing, nose poking or key pecking) and the delivery of reward. Second, the animal must be able to perceive and remember time. Third, the animal must act on its remembered time by starting and then stopping or inhibiting the response. Fourth, the animal must be able to compare the elapsed time in the trials with its remembered time to reinforcement. In each trial the time clock is reset, and the animal must reset its internal “counter,” i.e., at the beginning of each trial animals should start “timing” the trial time from zero. The ability to perform this task depends on the animal's working memory. Starting the internal clock at the beginning of the trial requires that the animal pays attention to the trial start time, which could be in the form of a visual signal, or as reported herein, the introduction of a lever into the experimental chamber. Failure to attend resulted in higher variability and a loss of accuracy during trial performance. Accuracy is measured by looking at the shape of the response function; therefore, if the response function is sharper and centered on the reinforcement time it supports a conclusion that attentional processes have been heightened.

All mice were food deprived to a target weight of 85-90% of their free-feeding weight before training began. Mice were fed approximately 10% of their body weight until they reached their target weight. On average, 1 week of food deprivation was sufficient to reach the target weight. During this time, all subjects were fed Bioserve 500 mg precision dustless pellets as their daily ration. They were exposed to Carnation™ evaporated milk in the home cage to avoid a possible neophobic reaction to the reinforcement. Subjects were given 1-week “vacations” every 4 to 6 weeks at which time they were allowed free access to food and a new free-feeding body weight baseline was recorded. Water was continuously available in the cage. For the amphetamine dose response curve, C57BL/6J mice were used; for the flesinoxan and 8-OH-DPAT studies, C3H mice were used.

Once deprived, animals were trained to lever-press in an operant chamber using ultrasensitive levers. Sixteen mouse operant chambers (Med Associates, Georgia, Vt.) were configured identically with two retractable ultra-sensitive levers, stimulus lights, and a dipper for delivery of condensed milk. A house light was placed on the opposite wall from the levers. Each chamber was placed in isolation cubicle. A fan provided white masking noise and adequate air.

Training and testing consisted of 1-h daily sessions. Subjects started on a concurrent fixed ratio 1-fixed time 1 min (FR 1-FT 1) schedule of reinforcement in which the house light served as the discriminative stimulus. Food was delivered every 1 min but the delivery was immediate if the animal made a response. Most animals acquired the lever-press response quickly, and those that did not were manually shaped by reinforcing successively closer approximations to the dipper using pinhole video cameras mounted in the attenuating cubicles. After no more than 1 week on this schedule, mice began training with a fixed-interval (FI) 10 s schedule in which all trials were separated by a 20 s, intertrial interval. The house light was on during the trials. Levers were introduced into the chamber at the beginning of the trial. All premature responses had no programmed consequences. Once a scalloped response curve was achieved, all subjects were placed on an FI 30 s schedule for approximately 1 week before moving to the peak interval procedure 30 second schedule (PIP 30 s).

Peak trials were programmed to occur at random with the restriction that no more than two unreinforced trials be presented consecutively. In peak trials, the house light was presented for 120 s. There was an average of eight peak trials per session. Responding was recorded in bins of 5 s each and monitored graphically. When the response rate showed a clear peak centered at 30 s, subjects were considered well trained. On average, it took 12 days for the mice to reach a clear peak. After about 3 weeks, mice were switched to a PIP 45 s, simply by changing reinforcement time and lever side. All other parameters were kept constant (intertrial interval, bin size, etc). As with the 30 s schedule, for the PIP 45 s mice were trained until stable performance was obtained. After 3 weeks in this condition, mice were switched to a double 30 s/45 s PIP procedure PIP 30 s-45 s: In this condition mice were tested with two different fixed interval values. The first half of the session consisted of either a PIP 30 s or a PIP 45 s, chosen at random. The second part of the session consisted of the other value. Therefore some mice started a session with a PIP 30 s and finished with a PIP 45 s, and some with the opposite order, but all experienced both orders across different days.

Once the performance during peak trials was stable, pharmacologic studies were commenced. During washout periods, all responses were unreinforced. Responses during these peak trials were recorded and transformed into a relative responding measure by dividing the number of responses in each 5 minute bin by the maximum response rate at any time interval in that trial. After relative responses had been calculated for each trial, an Analysis of Variance (ANOVA) with trial time and dose as within factors was performed. Significant interactions were followed up by planned pair-wise comparisons between the saline response and the corresponding drug dose response.

In subjects with problems of inhibition and response control, it will be beneficial to find a drug that improves performance by sharpening the response curve and providing the subject with greater control over the start and stop time for response. The experiments with mice and the timing procedure were designed to maximize the chance of finding drugs that improve performance. Amphetamine was tested in low to moderately high doses for comparison. Amphetamine, a drug of abuse, is used by humans to enhance attention or vigilance at low doses, and as a recreational drug (that results in a “high” state) at much higher doses (more than five times the attention enhancing dose).

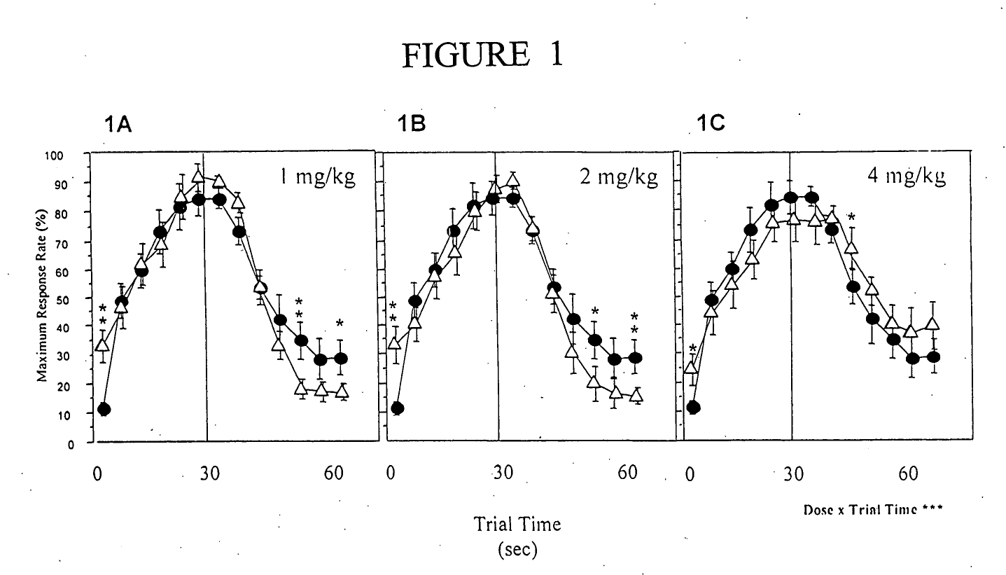

FIG. 1 shows the response pattern obtained with d-amphetamine. At the lower doses, 1 and 2 mg/kg, the amphetamine curve demonstrates a higher peak in the curve followed by a rapid decrease, relative to saline (FIGS. 1A, 1B). Conversely, at the higher 4 mg/kg dose, the amphetamine curve does not peak as high as the lower dose and the curve is flatter (FIG. 1C). Times at which pairwise comparisons between saline and amphetamine reached significance are indicated on the graphs. ANOVA revealed a significant dose×trial time interaction, p<0.001.

Improved performance is demonstrated by a sharp peak in the curve followed by a rapid decrease. A sharpened curve is indicative of heightened attentional processes. A less marked peak in the curve followed by a flattening is indicative of deteriorated performance. The lowest two doses of amphetamine (1 mg/kg, 2 mg/kg) had positive effects on the PIP 30 s task, as they sharpened the curve as depicted in FIGS. 1A and 1B. The highest dose of amphetamine (4 mg/kg) disrupted performance on the PIP 30 s task, flattening the curve as depicted in FIG. 1C. These results indicate that at low doses, amphetamine, a known therapeutic drug for ADHD, can improve performance in a task that measures attentive behavior.

### EXAMPLE 2

The following results were obtained using the methods described in Example 1, but with two different doses of the 5-HT_{1A} agonist flesinoxan (+)(4-fluoro-N-[2-[4-[2-(hydroxymethyl)-1,4-benzodioxane-5-yl]1-piperazinyl]ethyl]benzamide). After 4 weeks of training in the double PIP procedure, mice were tested with flesinoxan. Flesinoxan was dissolved in distilled H₂O and injected to half the mice in a low dose (0.1 mg/kg) or a lower dose (0.03 mg/kg), whereas the other subjects were injected with vehicle.

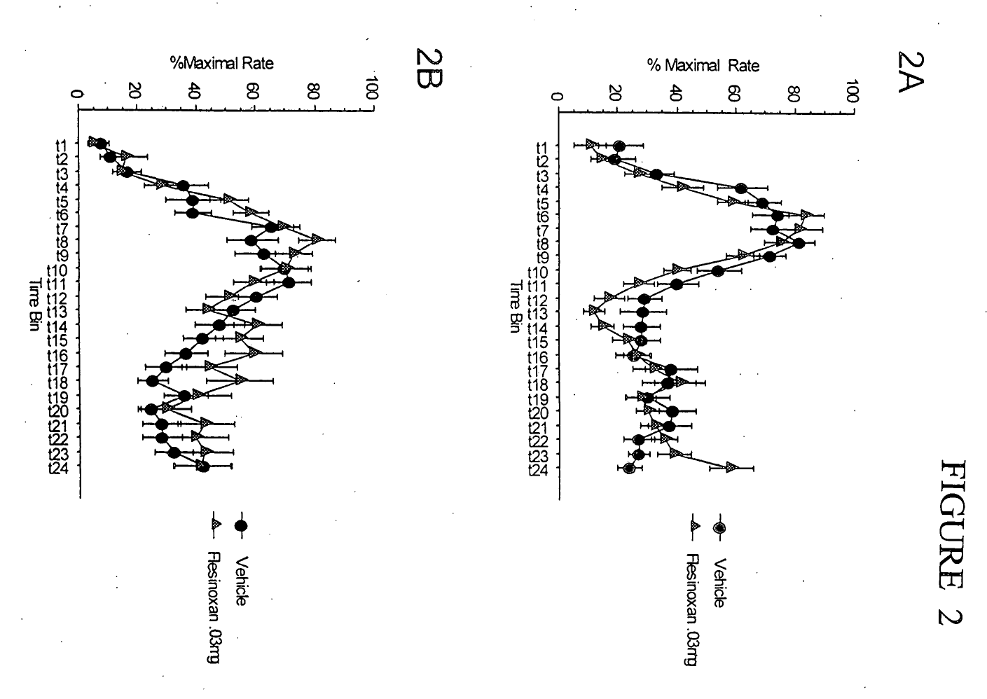

FIG. 2 demonstrates increased attentive behavior at the “lower” dose of flesinoxan. In the PIP 30 s, 0.03 mg/kg flesinoxan, the peaks of the response curve is higher and the curves are sharper than vehicle. This effect is evident but less robust in the PIP 45 s schedule, indicating that the more robust effect observed at the PIP 30 s could be attributable to a positive effect on attentional processes in contrast to a more central enhancing effect on information processing, which should result in better performance at all intervals tested. ANOVA revealed a significant dose×trial time interaction in the PIP 30 s (FIG. 2A; p<0.0023; F(1,23)=2.169), but not in the PIP45 s (FIG. 2B; p=0.9128; F(1,23)=0.619).

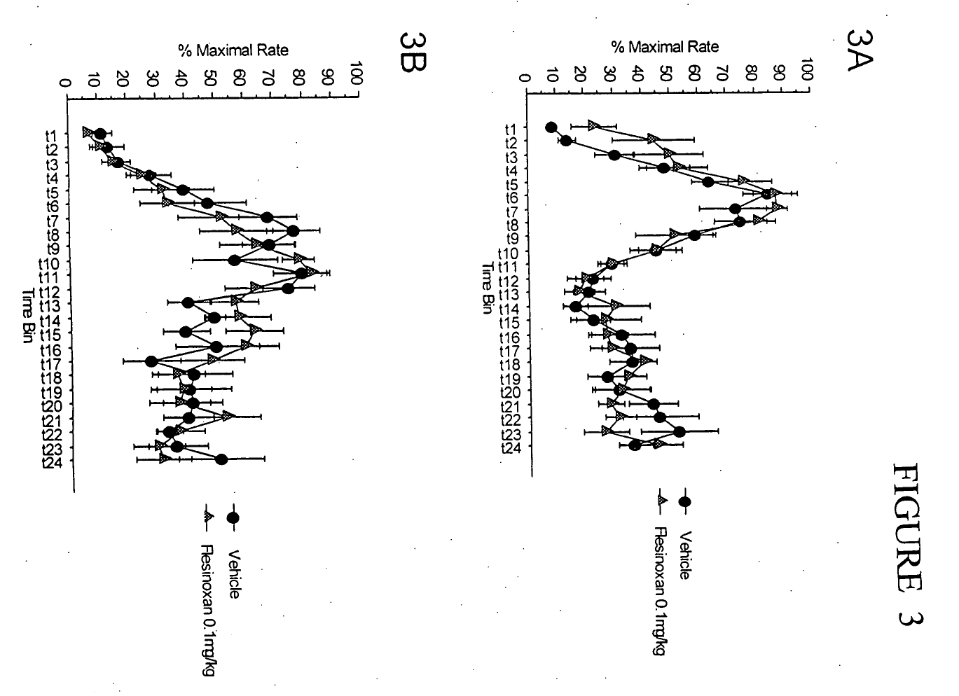

At a higher, but still relatively low, dose, however, flesinoxan was less effective. At 0.1 mg/kg flesinoxan, the performance curves were more comparable to saline curve both in the PIP 30 s and PIP 45 s (FIGS. 3A, and 3B, respectively), but were not disruptive (compare to FIG. 1C). ANOVA revealed no main dose effect or dose×trial time interaction. In conclusion, flesinoxan at low doses improves attentive behavior, and is expected to be useful in the treatment of ADHD.

### EXAMPLE 3

The following results were obtained using the methods described in Example 1. After 4 weeks of training in the double PIP procedure, mice were tested with the 5-HT_{1A} agonist 8-OH-DPAT. 8-OH-DPAT was dissolved in distilled H₂O and injected to half the mice in a low dose (0.1 mg/kg), whereas the other subjects were injected with vehicle. After 3 days of washout, the treatments were reversed and a Latin Square design was completed except that the mice previously treated with drug were treated with vehicle and those mice treated previously with vehicle were administered a lower dose of 8-OH-DPAT (0.01 mg/kg). After 2 days of washout, the Latin Square design was completed at the 0.01 mg/kg dose.

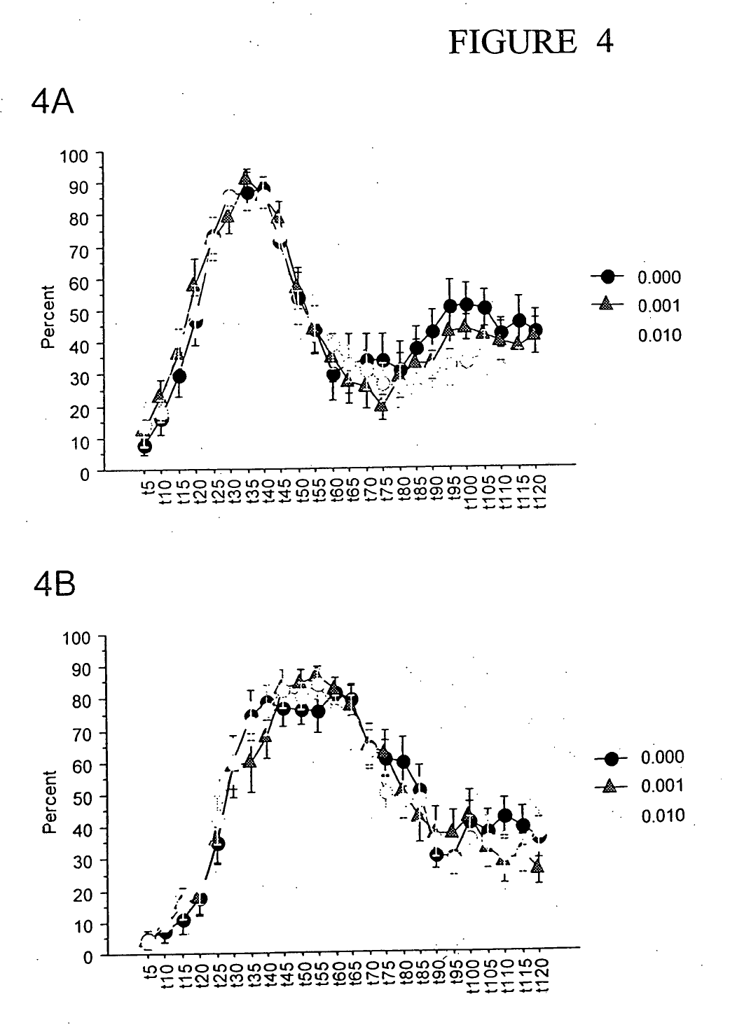

FIG. 4 demonstrates that at both 0.1 mg/kg and 0.01 mg/kg 8-OH-DPAT, the performance curves were not significantly different from the saline curve both in the PIP 30 s and PIP 45 s (FIGS. 4A and 4B, respectively). ANOVA revealed no dose main effect or dose×trial time interaction. The 0.01 mg/kg dose of 8-OH-DPAT was slightly disruptive.

In conclusion, 8-OH-DPAT was not shown to improve attentive behavior in the peak procedure. This result is most likely due to the short half-life of the compound.

### EXAMPLE 4

The coloboma (Cm) mutant mouse has been proposed as a rodent model for ADHD (for review, see Wilson, Neurosci. Biobehav. Rev., 24:51-57, 2000). The rationale for this proposal is three fold: first, Cm mutants (heterozygote) exhibit elevated spontaneous locomotor hyperactivity which averages three to four times the activity of wild-type littermates (Hess et al., J. Neurosci., 12:2865-2874, 1992; Hess et al. J. Neurosci., 16:3104-3111, 1996); second, this Cm mutation-associated hyperactivity can be ameliorated by low and moderate doses (2-16 mg/kg) of D-amphetamine (Hess et al., 1996, supra), a psychostimulant commonly prescribed to treat ADHD; and lastly, Cm mutant mice exhibit delays in achieving complex neurodevelopmental milestones in behavior (Heyser et al., Brain Res. Dev. Brain Res., 89:264-269, 1995) and deficits in hippocampal physiology and learning performance (Steffensen et al., Synapse, 22:281-289, 1996; Raber et al., J. Neurochem., 68:176-186, 1997) which may correspond to impairments seen in ADHD.

The genetic defects associated with Cm mutant mice include a deletion of the gene Snap (Hess et al., 1992, supra; Hess et al., Genomics, 21:257-261, 1994). Snap encodes SNAP-25, which is a key component of the synaptic vesicle docking and fusion complex required for regulated synaptic transmission. As a result, Cm mutant animals show marked deficits in Ca²⁺-dependent dopamine release (Raber et al., supra). This hypofunctioning DA system, which may involve meso-cortical, meso-limbic, as well as nigro-striatal circuitries has been suggested as a possible mechanism underlying hyperactivity associated with Cm mutation. (Sagvolden, et al., Behav. Brain Res., 94:61-71, 1998; Sagvolden and Sergeant, Behav. Brain Res., 94:1-10, 1998). Preliminary linkage studies suggest that polymorphs of SNAP-25 maybe associated with ADHD (Brophy et al., Mol. Psychiatr., 7:913-17, 2002; Barr et al., Mol. Psychiatr., 5:405-09, 2000). Cm mutant mice therefore may provide a useful animal model of ADHD.

Amphetamine, but not methylphenidate, normalizes the hyperactivity in Cm mutant mice; in both control and Cm mutants, methylphenidate increases locomotor activity in a dose-dependent manner (Hess et al., 1996, supra). The differential effect of these two ADHD medicaments, which both act at the presynaptic terminal, has been attributed to the differing mechanisms of action of increasing synaptic DA concentrations (Hess et al., 1996, supra).

It has now been surprisingly found that flesinoxan, a specific 5-HT_{1A} receptor agonist produces an amphetamine-like effect on hyperactivity in coloboma mice.

Animals

Heterozygote coloboma mice were originally purchased from The Jackson Laboratory (Bar Harbor, Me.) and were bred and maintained in our colony. In the current study, mutant mice and wild-type littermates, all aged 8 to 10 weeks, were used. Animals were divided into 4 groups: mutant/drug-treatment, mutant/vehicle-control, wild-type/drug-treatment, wild-type/vehicle-control (n=4-13 per group). Age and gender were balanced among groups. All animals were housed as littermates (2-4 mice per cage) and were maintained on ad libitum food and water with a 12 hr light/dark cycle.

Behavioral Testing

The Open-field (OF) test was performed under normal lighting conditions (400 lux). Mice (Cm/+, WT) were brought into the experimental room and allowed at least 1 hr of acclimation. Thirty min prior to testing, animals received an i.p. injection of either either d-amphetamine (4 mg/kg), saline vehicle, flesinoxan (0.3 mg/kg), or distilled H₂O vehicle. Each mouse was then placed into an OF arena (27×27×20 cm) with an infrared beam array system (Med Associates, St. Albans, Vt.) that automatically monitored the animal's activity. Four animals of matching genotype and treatment were tested at one time. The test session lasted 40 min and animals were returned to the home cage at the end of the session. Test measures included locomotion (distance traveled in cm) and number of center entries (zone crossings).

Results

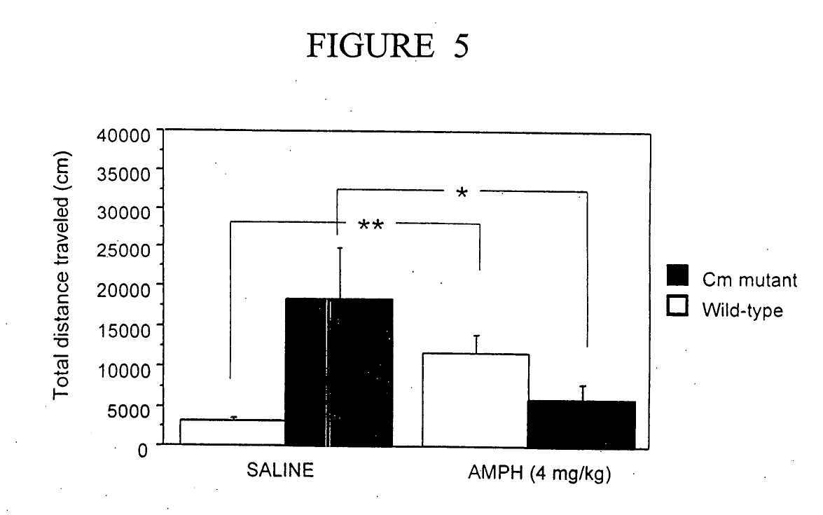

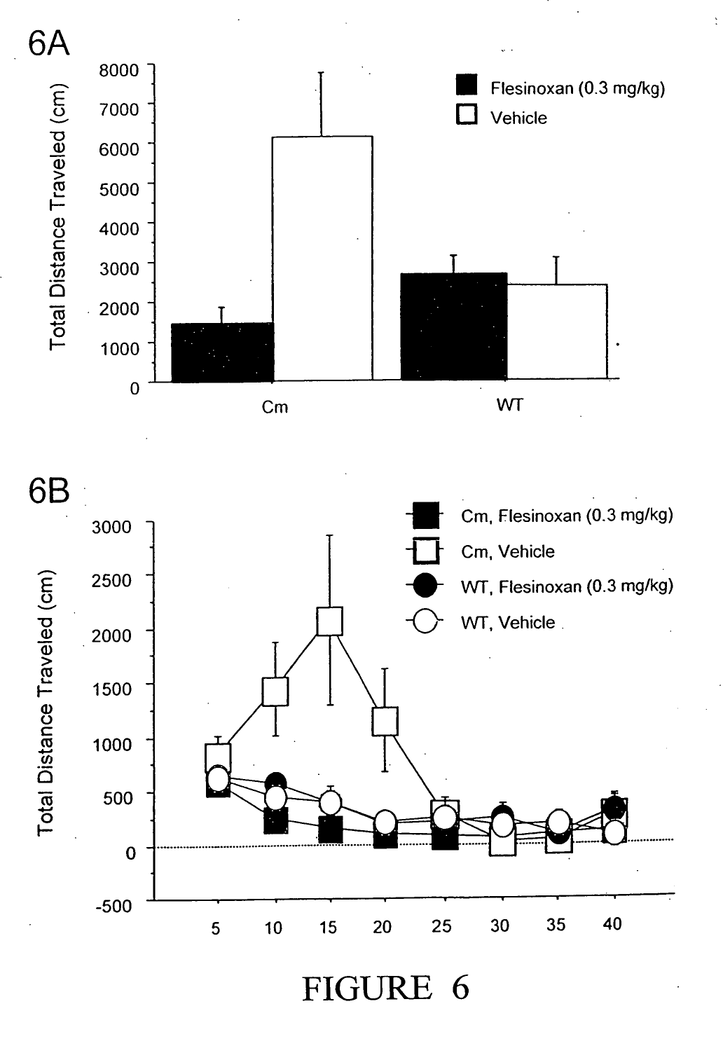

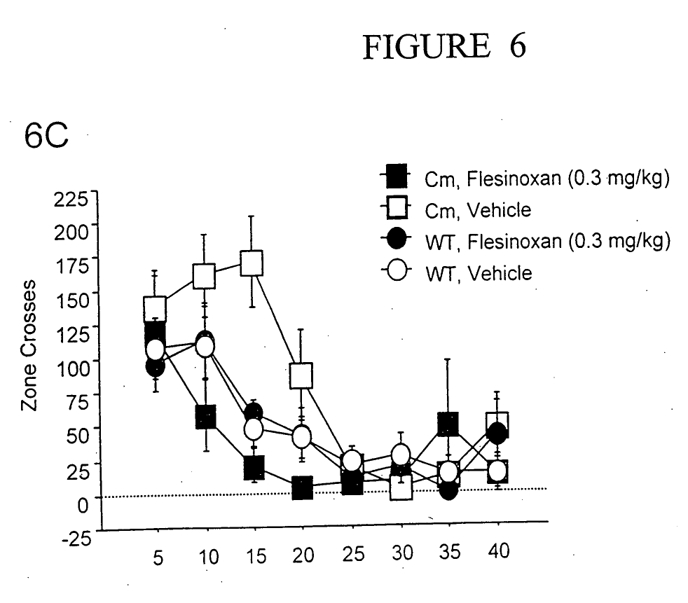

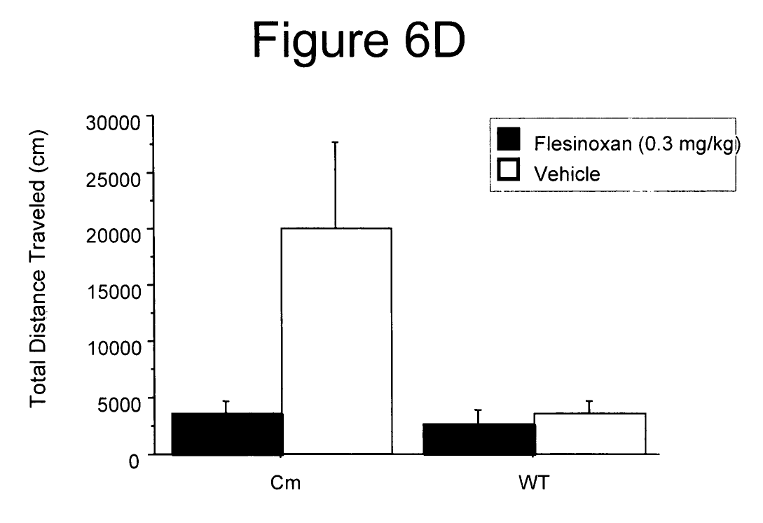

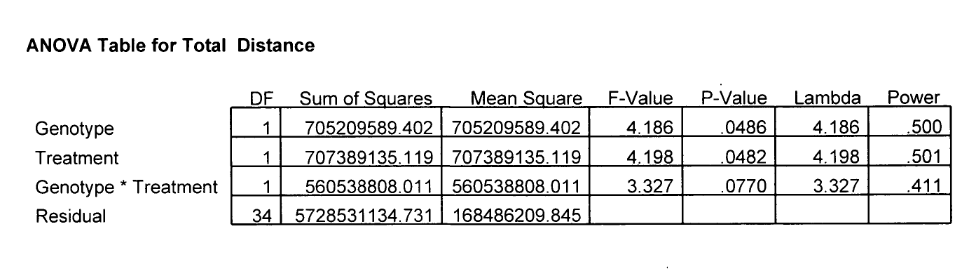

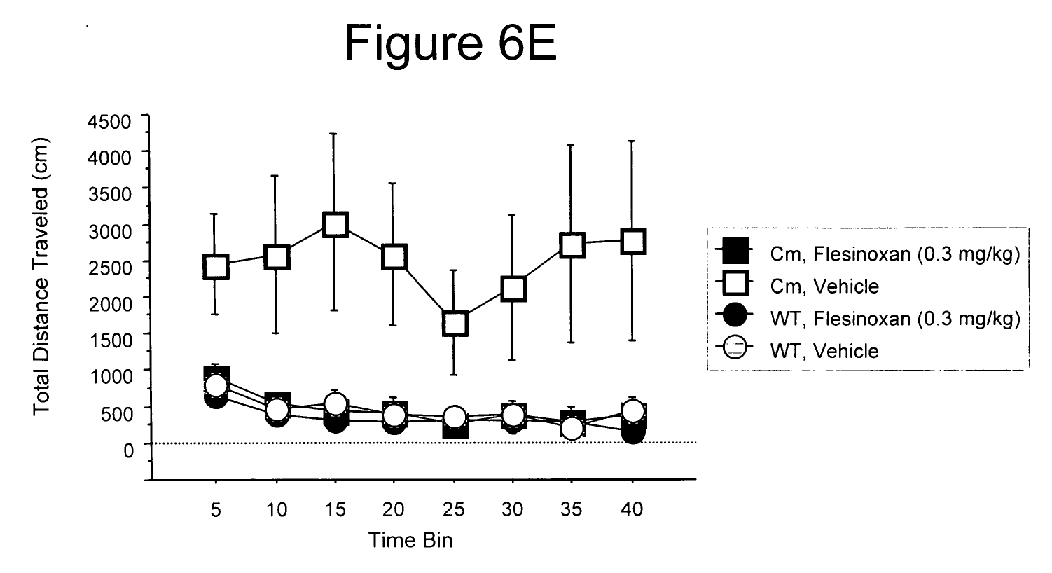

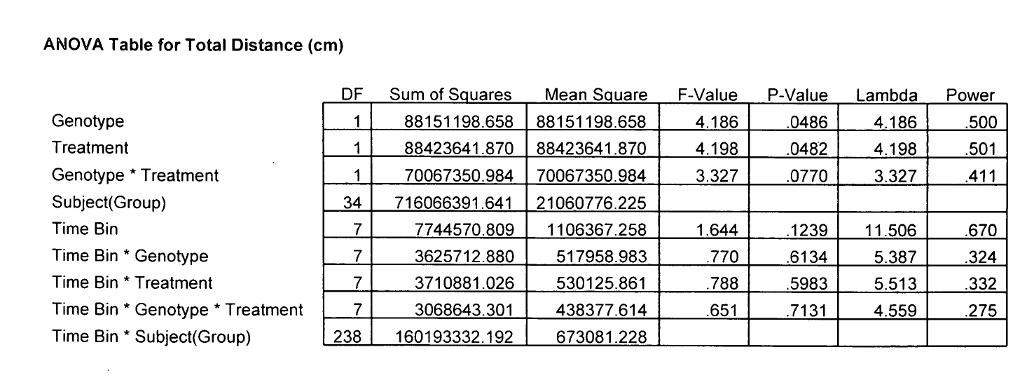

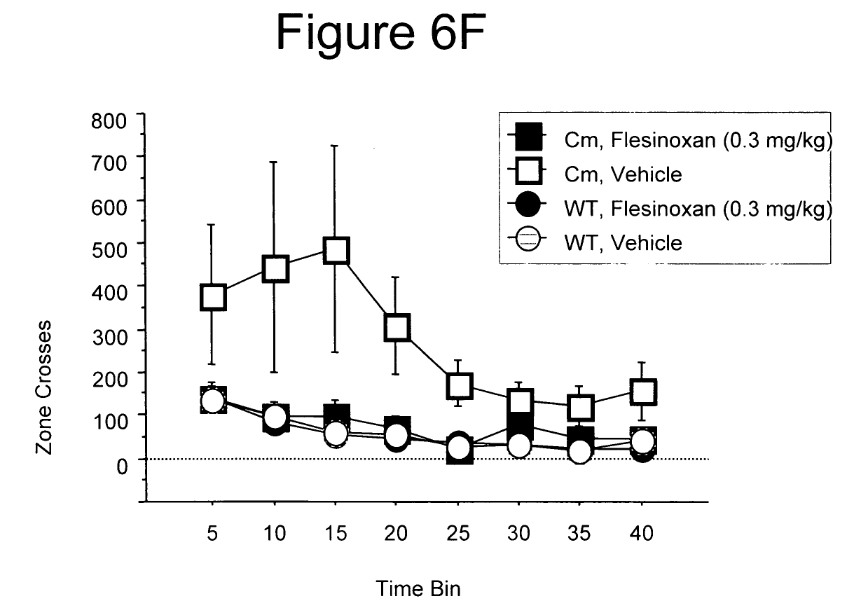

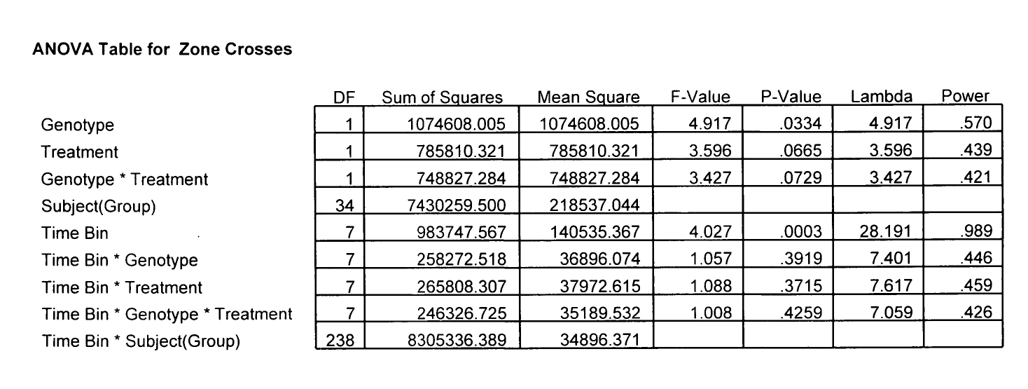

The data reveal a significant genotypic effect on parameters of hyperactivity that is reduced by amphetamine or flesinoxan treatment. Coloboma mutant mice are hyperactive relative to wild-type mice, as measured by increased locomotor activity. A genotypic effect on total ambulatory distance is depicted in FIGS. 5 and 6A, which illustrate that vehicle-treated mutant Cm mice traveled roughly three-six times further in distance than did their saline-treated wild-type littermates (18,386±6387 vs. 3116±338 centimeters, FIG. 5; 6112±1621 vs. 2385±692 centimeters, FIG. 6A, the scale of which is consistent with previously reported findings (Hess et. al., 1992, 1996, supra). And FIG. 6C, for example, illustrates that saline-treated mutant mice crossed zones more frequently than did their saline-treated wild-type littermates.

The genotype-related difference in locomotion was diminished in flesinoxan-treated animals and reversed in amphetamine-treated animals.

Administration of amphetamine, 4 mg/kg, to wild-type mice had a stimulatory effect, significantly increasing total distance traveled, as illustrated in FIG. 5 (3116±338 vs. 11657±2370 centimeters; ANOVA F_{(1,15)}=11.276, p=0.0043). By contrast, the same dose of amphetamine administered to Cm mutant mice significantly decreased total distance traveled relative to saline-treated Cm mutants (18386±6387 vs. 5966±1938 cm; ANOVA F_{(1,11)}=5.355, p=0.0459) to within the range of saline-treated wild-type mice. Amphetamine effectively normalized the hyperactive locomotor behavior of the coloboma mutant mice, significantly decreasing locomotion in the Cm mutant mice. ANOVA revealed a significant treatment×genotype interaction (F_{(1,25)}=11.038, p=0.0027).

Flesinoxan surprisingly had an amphetamine-like effect on total ambulatory distance of Cm mutant mice. Administration of 0.3 mg/kg flesinoxan reduced the total ambulatory distance of the Cm mutants from 6112±1621 to 1462±411, as illustrated in FIG. 6A. This represents a reduction in locomotor activity of more than 75% compared to that of saline-treated Cm mutants (F_{(1,20)}=5.138, p=0.034). ANOVA revealed a significant treatment×genotype effect (F_{(1,20)}=6.669, p=0.0178). Analyzed across 5 minute time bins, the effect of 0.3 mg/kg flesinoxan on Cm hyperactivity, as depicted in FIG. 6B, is equally impressive. ANOVA revealed a significant time×treatment interaction effect (F_{(1,140)}=3.901, p=0.006) time×genotype×treatment interaction effect (F_{(1,140)}=3.932, p=0.0006).

As FIG. 6C shows, flesinoxan decreased the frequency of zone crossings in Cm mutants to within the range of saline-treated wild-type mice, but the overall effect did not reach statistical significance (F_{(1,20)}=3.773, p=0.0683). When analyzed across 5 minute time bins, however, significant time×treatment (F_{(1,140)}=2.303, p=0.030) and time×treatment×genotype (F_{(1,140)}=3.626, p=0.0013) effects were observed.

Notably, flesinoxan only marginally affected locomotion in wild-type animals, as measured by distance traveled (FIGS. 6A and 6B) or zones crossed (FIG. 6C). This differential drug effect made the locomotor activity of flesinoxan-treated mutant and wild-type animals indistinguishable from that of saline-treated wild-type animals. In other words, flesinoxan effectively normalized the hyperactivity associated with the Cm mutation. FIGS. 6D-F represent the results of another trial conducted as described above, with the conclusions as indicated above.

It has previously been shown that the psychostimulant anti-ADHD agents d-amphetamine, but not methylphenidate, reinstated normal locomotor activity of the Cm mutants, suggesting an inconsistent effect of psychostimulants on this model of hyperactivity (Hess et. al., 1996, supra). However, the findings of this invention suggest that 5-HT_{1A} agonists, like flesinoxan have a specific regulatory role over hyperactivity. In summary, flesinoxan is acting like the anti-ADHD agent amphetamine in the Cm animal model of ADHD, but lacks the adverse stimulant properties of amphetamine observed in wild-type mice, demonstrating the therapeutic potential and advantages of 5-HT_{1A} agonist flesinoxan as anti-ADHD agents.

### EXAMPLE 5

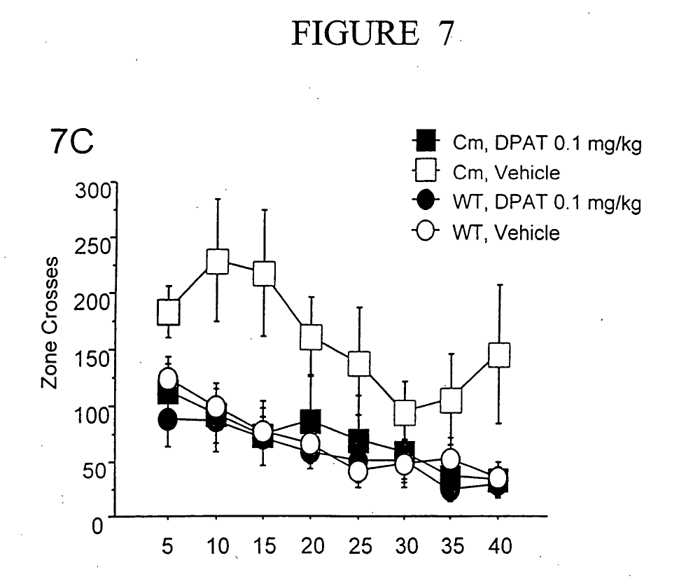

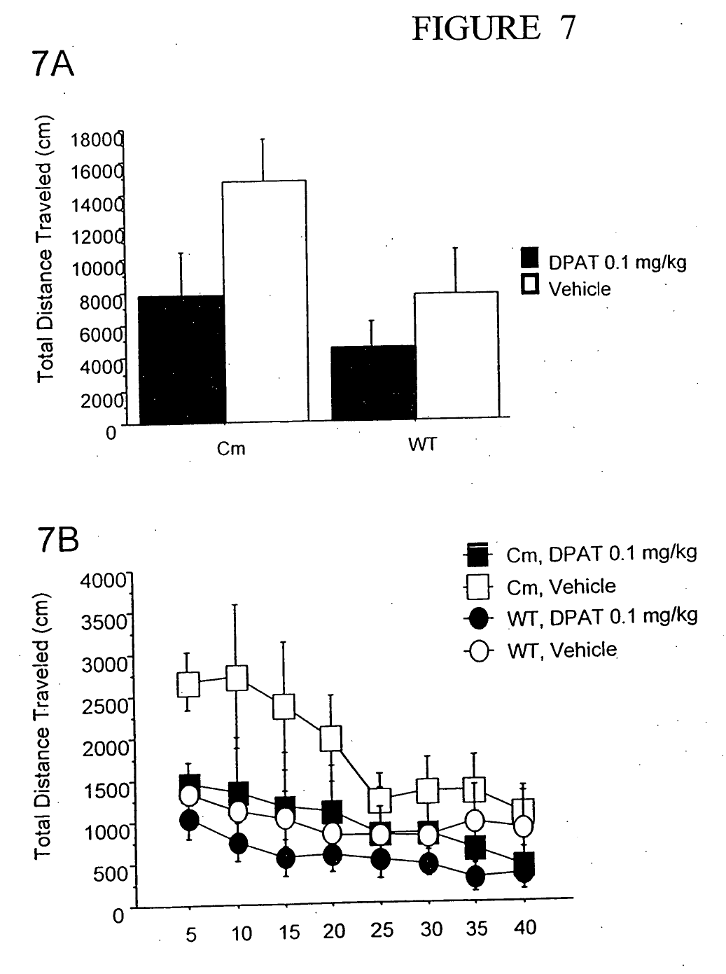

The following results were obtained using the methods described in Example 4. The Coloboma mutant (Cm) mice in this study exhibited hyperactivity that was surprisingly reduced by the 5-HT_{1A} agonist 8-OH-DPAT. Administration to Cm mice of 0.1 mg/kg of 8-OH-DPAT attenuated total ambulatory distance by almost 50% compared to vehicle: from 14702±2611 to 7813±2606 centimeters (FIG. 7A) This effect did not reach statistical significance, however, perhaps in part because of the high variability in the activity of the animals combined with the more limited hyperactivity of this group of Cm mutants (F_{(1,38})=3.914, p=0.552). When analyzed by 5 minute activity bins. (see FIG. 7B), the 8-OH-DPAT-induced reduction in hyperactivity also is evident, but the effect did not reach statistical significance. 8-OH-DPAT (0.1 mg/kg) significantly decreased the total frequency of zone crossings in Cm mice (FIG. 7C; F_{(1,38)}=5.098, p=0.030). ANOVA revealed no time treatment interaction effect, however. Lower doses of 8-OH-DPAT (0.001 or 0.01 mg/kg) produced no significant attenuation of Cm hyperactivity (data not shown), due in part to variability in activity among subjects.

In wild-type mice, 8-OH-DPAT decreased total ambulatory distance slightly, but non-significantly, from 7720±2726 to 4517±1599, as depicted in FIG. 7A; see also FIG. 7B. This differential drug effect made the overall locomotor activity of 8-OH-DPAT-treated mutant and wild-type animals indistinguishable from that of saline-treated wild-type animals. In other words, 8-OH-DPAT effectively normalized the hyperactivity associated with the Cm mutation. The findings of this invention suggest that 5-HT_{1A} agonists, like 8-OH-DPAT have a specific regulatory role over hyperactivity. In summary, 8-OH-DPAT is actin, like the anti-ADHD agent amphetamine in the Cm animal model of ADHD, demonstrating the therapeutic potential of 5-HT_{1A} agonists as anti-ADHD agents.

The above Examples are for illustrative purposes only and are not intended to limit the scope of the invention.

1. A method of treating Attention-Deficit/Hyperactivity Disorder (“ADHD”) in humans by administering a pharmaceutical formulation containing a therapeutically effective amount of a compound or compounds having full agonist or partial agonist activity at 5-HT_{1A} receptors, wherein any non-5-HT_{1A} agonist that is included in said formulation as an active ingredient, no such active ingredient is nicotine or a nicotinic agonist with the proviso that said formulation does not comprise nicotine or a nicotine agonist or buspirone or sunipetron.

2. A method according to claim 1 wherein 5-HT_{1A} agonist is a compound according to the formula I: 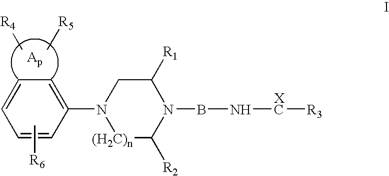
wherein
R₁ and R₂ independently of each other represent hydrogen or an alkyl having 1-3 carbon atoms;
R₃ is an aryl group or heteroaryl group which may be substituted with one or more substituents selected from the group consisting of halogen, trifluoromethyl, nitrile, nitro, alkoxy, having 1-3 carbon atoms, hydroxy, esterified hydroxy, and alkyl having 1 or 2 carbon atoms;
X is O, S, or NH;
B is the group —CH₂—CH₂— or —CH(CH₃)—CH₂—;
n has the value 0 or 1;
p has the value 0 or 1;
where p has the value 1,
A is O—CH₃, or forms, with the two carbon atoms of the phenyl group, an optionally substituted, entirely or partly unsaturated, cyclic group having 5-7 atoms in the ring, which comprises 1-3 hetero atoms from the group O, S, and N, with the proviso that the sum of the number of oxygen and sulfur atoms is at most two,
and where A is not O—CH₃,
R₄ is hydrogen or straight or branched chain alkyl having 1-3 carbon atoms and R₅ is hydrogen, halogen, alkyl having 1-3 carbon atoms, methylene, ethyldiene or vinyl, a straight or branched hydroxyalkyl group having 1-3 carbon atoms, which may be etherified or esterified, or an alkyl branched hydroxyalkyl group having 1-3 carbon atoms in the straight or branched alkyl group, an oxo group or a phenyl group; and
R₆ is a hydrogen or fluoro atom;
wherein
the compound may be a racemate or a single diastereomer or enantiomer;
or a pharmaceutically acceptable acid addition salt thereof.

3. The method of claim 2,
wherein
R₁, R₂, and R₆ are hydrogen;
R₃, is a lipophilic aromatic alkyl, selected from the group consisting of benzene, halogenated benzene, cyclohexane, and 2-thiophene;
X is O, S, or NH;
B is the group —CH₂—CH₂—
n has the value 1; and
p has the value 0 or 1,
and where p has the value 1,
A is O—CH₃, or forms, with the two carbon atoms of the phenyl group, an optionally substituted benzodioxane, a hydroxyalkyl having 1-2 carbon atoms, or a furan, R₃,
and where A is not O—CH₃,
R₄ is hydrogen, and
R₅ is hydrogen, or chiral —CH₂OH— at the 2 position of the benzodioxane ring.

4. The method according to claim 3, w % herein the compound is flesinoxan, wherein
R₁, R₂, and R₆ are hydrogen;
R₃, is halogenated benzene group, having a fluoro in the para position;
X is O;
B is the group —CH₂—CH₂—;
n has the value 1;
p has the value 1;
A is benzodioxane;
R₄ is hydrogen
R₅ is chiral —CH₂OH— at the 2 position of the benzodioxane ring; and
the salt is hydrochloride.

5. The method according to claim 4, wherein the compound is administered at a dose of approximately 0.04 mg/day to 4 mg/day.

6. The method according to claim 5, wherein the compound is administered at a dose of approximately 0.1 mg/day and 1 mg/day.

7. The method according to claim 6, wherein the compound is administered at a dose of approximately 0.1 mg/day and 0.5 mg/day.

8. The method according to claim 1, wherein the 5-HT_{1A} agonist is a compound having the formula II: 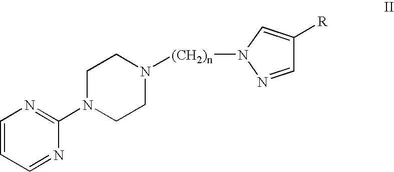
wherein
n can have the value 1 to 6;
R is a hydrogen, a halogen, a lower alkyl radical having 1-4 carbon atoms, a heteroaryl radical, a sulpho radical, an N-substituted or N,N-disubstituted sulphamoyl radical, a nitro radical, a hydroxyl radical, an oxo radical, a lower alkoxyradical having 1-4 carbon atoms, a cyano radical, a lower alkylcarboxylate radical having 1-4 carbon atoms, an aryl or substituted aryl radical, or an amino or substituted amino radical of formula 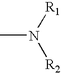
in which R₁ and R₂, independently are a hydrogen, an alkyl radical, an aryl radical, an alkylcarbonyl radical, an arylcarbonyl radical, an alkylsulphonyl radical or an arylsulphonyl radical, the alkyl fragments of these radicals containing from 1-4 carbon atoms; and
wherein
the compound may be a racemate or a single diastereomer or enantiomer;
or a pharmaceutically acceptable acid addition salt thereof.

9. The method according to claim 8, wherein the compound is lesopitron,
wherein
n is 4,
R is chloro; and
the salt is dihydrochloride.

10. The method according to claim 1, wherein the 5-HT_{1A} agonist is selected from the group consisting of flesinoxan, lesopitron, BAY x 3702, F11440, LY228729, LY293284, NAE-086, S14506, S14671, S16924, or gepirone.

11. The method according to claim 1, wherein the 5-HT_{1A} agonist(s) is the sole ADHD active component(s) of the formulation.

12. The method according to claim 10, wherein the 5-HT_{1A} agonist is administered at a dose of approximately 0.01 mg/day to 100 mg/day.

13. The method according to claim 10, wherein the 5-HT_{1A} agonist is administered at a dose of approximately 0.1 mg/day and 10 mg/day.

14. The method according to claim 10, wherein the 5-HT_{1A} agonist is administered at a dose of approximately 0.1 mg/day and 2 mg/day.

15. The method according to claim 1, wherein the intrinsic activity of the 5-HT_{1A} agonist is at least 0.5-1.0.

16. The method according to claim 15, wherein the intrinsic activity of the 5-HT_{1A} agonist is at least about 0.6-1.0.

17. The method according to claim 16, wherein the intrinsic activity of the 5-HT_{1A} agonist is at least about 0.7-1.0.

18. The method according to claim 17, wherein the intrinsic activity of the 5-HT_{1A} agonist is at least about 0.8-1.0.

19. The method according to claim 2, wherein the difference in affinity of the 5-HT_{1A} agonist for 5-HT_{1A} receptors compared to any of 5-HT_{1B/1D}, 5-HT₂, D₂, D₄, α₁ or α₂ receptors or SERT, DAT, or NET (ΔpKᵢ) is at least 1.

20. The method according to claim 2, wherein the difference in affinity of the 5-HT_{1A} agonist for 5-HT_{1A} compared to D₂ receptors (ΔpKᵢ) is at least about 2.

21. The method according to claim 2, wherein the difference in affinity of the 5-HT_{1A} agonist for 5-HT_{1A} receptors compared to any of 5-HT_{1B/1D}, 5-HT₂, D₂, D₄, α₁ or α₂ receptors or SERT, DAT, or NET (ΔpKᵢ) is at least about 2.
